(19) United States
(12) Patent Application Publication
(10) Pub. No.: US 2024/0298895 A1
(43) Pub. Date: Sep. 12, 2024

(54) BLOOD PRESSURE MEASUREMENT WITH HAPTIC CALIBRATION

(71) Applicant: Whoop, Inc., Boston, MA (US)

(72) Inventor: Mostafa Ghannad-Rezaie, Malden, MA (US)

(21) Appl. No.: 18/601,457

(22) Filed: Mar. 11, 2024

Related U.S. Application Data
(60) Provisional application No. 63/451,193, filed on Mar. 9, 2023.
(60) Provisional application No. 63/596,469, filed on Nov. 6, 2023.

(51) Int. Cl. A61B 5/00

(52) U.S. Cl. CPC A61B 5/0051; A61B 5/6824; A61B 5/6831; A61B 2562/0219

(57) ABSTRACT

A wearable physiological monitor is configured through a calibration procedure to provide a calibrated blood pressure measurement based on signals from an optical sensing system. In one aspect, optical (PPG) signals and motion signals are acquired while applying a mechanical stimulus over a range of mechanical frequencies with a haptic actuator. Resulting data is used to create a dynamic model for calculating blood pressure based on motion of the monitor. This blood pressure measurement can also usefully be correlated to the PPG signal for continuous blood pressure estimation. In another aspect, a monitoring device is positioned over a radial artery, and then optical measurements are taken of the radial artery while varying (and measuring) an applied force. Using various techniques, a model can be derived from this data for continuous blood pressure estimation.

### RELATED APPLICATIONS

This application claims priority to U.S. Prov. App. No. 63/451,193 filed on Mar. 9, 2023 and U.S. Prov. App. No. 63/596,469 filed on Nov. 6, 2023. This application is also related to Int'l App. No. PCT/US24/19290 filed on Mar. 9, 2024 and Int'l App. No. PCT/US24/19381 filed on Mar. 11, 2024. The entire contents of each of the foregoing applications is hereby incorporated by reference.

### BACKGROUND

There remains a need for techniques to measure blood pressure with a continuously wearable monitor.

### SUMMARY

A wearable physiological monitor is configured through a calibration procedure to provide a calibrated blood pressure measurement based on signals from an optical sensing system. In one aspect, optical (PPG) signals and motion signals are acquired while applying a mechanical stimulus over a range of mechanical frequencies with a haptic actuator. Resulting data is used to create a dynamic model for calculating blood pressure based on motion of the monitor. This blood pressure measurement can also usefully be correlated to the PPG signal for continuous blood pressure estimation. In another aspect, a monitoring device is positioned over a radial artery, and then optical measurements are taken of the radial artery while varying (and measuring) an applied force. Using various techniques, a model can be derived from this data for continuous blood pressure estimation.

In one aspect, a method disclosed herein includes acquiring a first optical signal from a wearable device while the wearable device is placed for use on a body of a user; applying a mechanical pressure to the wearable device with a mechanical actuator; acquiring a second optical signal from the wearable device while applying the mechanical pressure; acquiring a motion signal from the wearable device, at least a portion of the motion signal acquired while applying the mechanical pressure; based on the first optical signal, the second optical signal, and the motion signal, calculating one or more physical parameters of a dynamic model for a mechanical system including the wearable device, a strap securing the wearable device to the user, and the mechanical actuator; calculating a blood pressure of the user by applying the motion signal to the dynamic model; and displaying the blood pressure to a user.

The wearable device may be a physiological monitor, such as a photoplethysmography monitor or a heart rate monitor. The wearable device may include a wrist worn monitor with a strap. The one or more physical parameters may include a tension of a strap securing the wearable device to the user. The one or more physical parameters may include an elasticity of a tissue of the user in a measurement volume of the wearable device. The one or more physical parameters may include a spring constant for a system including the wearable device and a strap securing the wearable device to the user. The one or more physical parameters may include an elasticity of a tissue of the user adjacent to the wearable device. The mechanical actuator may include a haptic output device associated with the wearable device.

Applying mechanical pressure may include vibrating the wearable device with a haptic stimulus. The haptic stimulus may include a time varying haptic vibration. The haptic stimulus may include a haptic CHIRP output. The motion signal may include data from an accelerometer. The motion signal may include data from at least one of a three-axis accelerometer and a three-axis gyroscope. Calculating one or more physical parameters may include evaluating at least one of the physical parameters with a machine learning model based on inputs from one or more sensors of the wearable device. At least one of the first optical signal and the second optical signal may be acquired when the user is at rest. At least one of the first optical signal and the second optical signal may include a photoplethysmography signal. The method may include testing tension on a strap of the wearable device by performing the steps of: acquiring a baseline optical signal; applying a haptic stimulus after acquiring the baseline optical signal; acquiring a test optical signal during the haptic stimulus; and analyzing the test optical signal for motion artifacts indicative of a suitable tension range for measuring blood pressure with the wearable device. The method may further include calculating a change in blood pressure based on a change in a pulse shape acquired by the wearable device from a first pulse acquired temporally proximal to measuring the motion signal from the wearable device to a second pulse acquired temporally distal to measuring the motion signal; and calculating a blood pressure for the user based on the calibrated blood pressure and the change in blood pressure.

In another aspect, a computer program product disclosed herein includes computer executable code embodied in a non-transitory computer readable medium that, when executing on one or more devices, causes the one or more devices to perform the steps of: acquiring a first optical signal from a wearable device; applying a mechanical pressure to the wearable device with a mechanical actuator while the wearable device is placed for use on a body of a user; acquiring a second optical signal and a motion signal from the wearable device while applying the mechanical pressure; based on the optical signals and the motion signal, calculating one or more parameters of a model for a system including the wearable device, a strap securing the wearable device to the user, and the mechanical actuator; calculating a blood pressure of the user with the motion signal and the model; and displaying the blood pressure to a user.

In another aspect, a method disclosed herein includes calibrating a model for a wearable device while the wearable device is placed for use on a user, the wearable device including a haptic actuator and a motion sensor, and the model expressing a calibrated blood pressure for a wearer of the wearable device over time as a function of a number of constants and a motion signal for the wearable device over time; operating the haptic actuator with a driving function; measuring the motion signal for the wearable device from the motion sensor while operating the haptic actuator; and calculating the calibrated blood pressure with the model based on the motion signal measured while operating the haptic actuator.

The method may include analyzing the calibrated blood pressure over one or more time intervals to identify a systolic blood pressure and a diastolic blood pressure for the user. The method may include correlating the calibrated blood pressure to a photoplethysmography signal from the wearable device; and calculating a second blood pressure for the user based on the calibrated blood pressure and a second photoplethysmography signal acquired after operating the haptic actuator and measuring the motion signal. The method may include analyzing the second blood pressure over one or more time intervals to identify a systolic blood pressure and a diastolic blood pressure for the user over the one or more intervals.

The number of constants may include one or more of a tension of a strap securing the wearable device to the user, a spring constant for the strap securing the wearable device to the user, and an elasticity for tissue of the user in a measurement volume for the wearable device. Calibrating the model may include calculating one or more of the number of constants. The wearable device may include a wearable photoplethysmography monitor. The wearable device may include a heart rate monitor. The motion sensor may include an accelerometer. The motion sensor may include a gyroscope. The method may include calculating a blood pressure for the user based on the calibrated blood pressure and a change in a pulse shape acquired by the wearable device from a first pulse acquired temporally proximal to measuring the motion signal from the wearable device to a second pulse acquired temporally distal to measuring the motion signal.

In another aspect, a computer program product disclosed herein includes computer executable code embodied in a non-transitory computer readable medium that, when executing on one or more devices, causes the one or more devices to perform the steps of: calibrating a model for a wearable device while the wearable device is placed for use on a user, the wearable device including a haptic actuator and a motion sensor, and the model expressing a calibrated blood pressure for a wearer of the wearable device over time as a function of a number of constants and a motion signal for the wearable device over time; operating the haptic actuator with a driving function; measuring the motion signal for the wearable device from the motion sensor while operating the haptic actuator; and calculating the calibrated blood pressure with the model based on the motion signal measured while operating the haptic actuator. The computer executable code may cause the one or more devices to perform the steps of: calculating a change in blood pressure based on a change in a pulse shape acquired by the wearable device from a first pulse acquired temporally proximal to measuring the motion signal from the wearable device to a second pulse acquired temporally distal to measuring the motion signal; and calculating a blood pressure for the user based on the calibrated blood pressure and the change in blood pressure.

In another aspect, a system disclosed herein includes a wearable device including a strap for coupling the wearable device to a user, a haptic output device, and a plurality of motion sensors; a memory storing a physical model for the wearable device, the physical model calibrated with the wearable device secured to the user with the strap, and the physical model expressing a calibrated blood pressure for a wearer of the wearable device over time as a function of at least a number of constants and a motion signal for the wearable device over time; and a processor configured by computer executable code stored in a non-transitory computer readable medium to perform the steps of: operating the haptic output device with a driving function; measuring the motion signal for the wearable device while operating the haptic output device; calculating the calibrated blood pressure with the physical model over a period of time based on the motion signal measured while operating the haptic output device; and analyzing the calibrated blood pressure over the period of time to identify a systolic blood pressure and a diastolic blood pressure for the user. The process may be further configured to perform the steps of calculating a change in blood pressure based on a change in a pulse shape acquired by the wearable device from a first pulse acquired temporally proximal to measuring the motion signal from the wearable device to a second pulse acquired temporally distal to measuring the motion signal; and calculating a blood pressure for the user based on the calibrated blood pressure and the change in blood pressure.

In another aspect, a computer program produce disclosed herein includes computer executable code embodied in a non-transitory computer readable medium that, when executing on one or more devices, causes the one or more devices to perform the steps of: measuring a calibrated blood pressure of a user in mmHG using a haptic actuator of a wearable monitor, the calibrated blood pressure including a systolic blood pressure and a diastolic blood pressure; acquiring a first PPG signal from the user with the wearable monitor, the first PPG signal temporally proximal to measuring the calibrated blood pressure, and the first PPG signal including a first pulse; associating a first shape of the first pulse with the calibrated blood pressure; acquiring a second PPG signal from the user with the wearable monitor, the second PPG signal including a second pulse temporally distal to measuring the calibrated blood pressure; detecting a change in pulse shape from the first shape of the first pulse to a second shape of the second pulse; estimating a change in blood pressure from the calibrated blood pressure based on the change in pulse shape, the change in blood pressure including a first change in the systolic blood pressure and a second change in the diastolic blood pressure; and calculating an estimated blood pressure for the user based on the calibrated blood pressure and the estimated change in blood pressure.

In another aspect, a method disclosed herein includes measuring a calibrated blood pressure of a user in mmHG; acquiring a first PPG signal from the user with the wearable monitor, the first PPG signal temporally proximal to measuring the calibrated blood pressure, and the first PPG signal including a first pulse; associating a first shape of the first pulse with the calibrated blood pressure; acquiring a second PPG signal from the user with the wearable monitor, the second PPG signal including a second pulse temporally distal to measuring the calibrated blood pressure; detecting a change in pulse shape from the first shape of the first pulse to a second shape of the second pulse; estimating a change in blood pressure from the calibrated blood pressure based on the change in pulse shape; and calculating an estimated blood pressure for the user based on the calibrated blood pressure and the estimated change in blood pressure.

Detecting the change in pulse shape may include measuring, for the first PPG signal and the second PPG signal, at least one of a height of a pulse dicrotic notch, a slope, a height of a systolic peak, a height of a diastolic peak, an interval between the systolic peak and the dicrotic notch, a pulse width, a pulse area, and a peak-to-peak interval. Detecting the change in pulse shape may also or instead include measuring, for the first PPG signal and the second PPG signal, at least one of an augmentation index and an arterial stiffness index.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a device for wearable physiological monitoring.

FIG. 2 is a block diagram of a computing device that may be used herein.

FIG. 3 illustrates a physiological monitoring system.

FIG. 4 is a flow chart illustrating a method for measuring fit of a wearable monitor and providing actionable feedback to a user.

FIG. 5 is a flow chart illustrating a method for measuring fit of a wearable monitor and providing actionable feedback to a user.

FIG. 6 illustrates a coordinate system for measuring device motion.

FIG. 7 illustrates a mapping of mechanical and optical coupling to device fit.

FIG. 8 illustrates a user interface for user interactions with a device fit protocol.

FIG. 9 shows motion data from a wearable monitor.

FIG. 10 shows a confusion matrix for data-driven predictions of body location for a wearable monitor.

FIG. 11 illustrates the time to achieve a 95% confidence level in location predictions for a wearable monitor on a number of body locations.

FIG. 12 is a flow chart illustrating a method for creating a model to calculate calibrated blood pressure.

FIG. 13 is a flow chart illustrating a method for using a model to calculate calibrated blood pressure from an optical signal.

FIG. 14 is a flow chart illustrating a method for measuring blood pressure.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

Exemplary embodiments provide physiological measurement systems, devices and methods for continuous health and fitness monitoring, and provide improvements to overcome the drawbacks of conventional heart rate monitors. One aspect of the present disclosure is directed to providing a lightweight wearable system with a strap that collects various physiological data or signals from a wearer. The strap may be used to position the system on an appendage or extremity of a user, for example, wrist, ankle, and the like. Exemplary systems are wearable and enable real-time and continuous monitoring of heart rate without the need for a chest strap or other bulky equipment which could otherwise cause discomfort and prevent continuous wearing and use. The system may determine the user's heart rate without the use of electrocardiography and without the need for a chest strap. Exemplary systems can thereby be used in not only assessing general well-being but also in continuous monitoring of fitness. Exemplary systems also enable monitoring of one or more physiological parameters in addition to heart rate including, but not limited to, body temperature, heart rate variability, motion, sleep, stress, fitness level, recovery level, effect of a workout routine on health and fitness, caloric expenditure, and the like.

A health or fitness monitor that includes bulky components may hinder continuous wear. Existing fitness monitors often include the functionality of a watch, thereby making the health or fitness monitor quite bulky and inconvenient for continuous wear. Accordingly, one aspect is directed to providing a wearable health or fitness system that does not include bulky components, thereby making the bracelet slimmer, unobtrusive and appropriate for continuous wear. The ability to continuously wear the bracelet further allows continuous collection of physiological data, as well as continuous and more reliable health or fitness monitoring. For example, embodiments of the bracelet disclosed herein allow users to monitor data at all times, not just during a fitness session. In some embodiments, the wearable system may or may not include a display screen for displaying heart rate and other information. In other embodiments, the wearable system may include one or more light emitting diodes (LEDs) to provide feedback to a user and display heart rate selectively. In some embodiments, the wearable system may include a removable or releasable modular head that may provide additional features and may display additional information. Such a modular head can be releasably installed on the wearable system when additional information display is desired and removed to improve the comfort and appearance of the wearable system. In other embodiments, the head may be integrally formed in the wearable system.

Exemplary embodiments also include methods for measuring tightness of a wearable monitor and providing actionable feedback to a user. The tightness of the wearable monitor may have an impact on its performance. To help ensure a good fit, a physical model such as a spring model or resonance model may be created to characterize movement of the wearable monitor when elastically retained in tension about a body part. The wearable monitor may then be vibrated, and a response to these vibrations may be applied to the model to infer the tension. The inferred tension may be used to provide adjustment information to the user.

The term “continuous,” as used herein in connection with heart rate data collection, refers to collection of heart rate data at a sufficient frequency to enable detection of individual heartbeats, and also refers to collection of heart rate data continuously throughout the day and night. More generally with respect to physiological signals that might be monitored by a wearable device, “continuous” or “continuously” will be understood to mean continuously at a rate suitable for intended time-based processing, and physically at a rate possible by the monitoring hardware, subject to ordinary data acquisition limitations such as sampling limitations and sampling rates associated with converting physical signals into digital data, and physical limitations associated with physical disruptions during use, e.g., temporary displacement of monitoring hardware due to sudden movements, changes in external lighting, loss of electrical power, physical manipulation or adjustment by a wearer, physical displacement of monitoring hardware due to external forces, and so forth. It will also be noted that heart rate data or a monitored heart rate, in this context, may more generally refer to raw sensor data, heart rate data, signal peak data, heart rate variability data, or any other physiological or digital signal suitable for recovering heart rate data as contemplated herein, and that heart rate data may generally be captured over some historical period that can be subsequently correlated to various metrics such as sleep states, activity recognition, resting heart rate, maximum heart rate, and so forth.

The term “pointing device,” as used herein, refers to any suitable input interface, specifically, a human interface device, that allows a user to input spatial data to a computing system or device. In an exemplary embodiment, the pointing device may allow a user to provide input to the computer using physical gestures, for example, pointing, clicking, dragging, and dropping. Exemplary pointing devices may include, but are not limited to, a mouse, a touchpad, a touchscreen, and the like.

The term “computer-readable medium,” as used herein, refers to a non-transitory storage hardware, non-transitory storage device or non-transitory computer system memory that may be accessed by a controller, a microcontroller, a computational system or a module of a computational system to encode thereon computer-executable instructions or software programs. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM) and the like.

The term “distal,” as used herein, refers to a portion, end or component of a physiological measurement system that is farthest from a user's body when worn by the user.

The term “proximal,” as used herein, refers to a portion, end or component of a physiological measurement system that is closest to a user's body when worn by the user.

The term “equal,” as used herein, refers, in a broad lay sense, to exact equality or approximate equality within some tolerance.

Exemplary embodiments provide wearable physiological measurements systems that are configured to provide continuous measurement of physiological data such as heart rate or other physiological data such as blood pressure, hydration state, blood oxygenation state, etc. Exemplary systems are configured to be continuously wearable on an appendage, for example, wrist or ankle, and do not rely on electrocardiography or chest straps in detection of heart rate. The exemplary system includes one or more light emitters for emitting light at one or more desired frequencies toward the user's skin, and one or more light detectors for received light reflected from the user's skin. The light detectors may include a photoresistor, a phototransistor, a photodiode, and the like. As light from the light emitters (for example, green light) pierces through the skin of the user, the blood's natural absorbance or transmittance for the light provides fluctuations in the photo-resistor readouts. These waves have the same frequency as the user's pulse since increased absorbance or transmittance occurs only when the blood flow has increased after a heartbeat. The system includes a processing module implemented in software, hardware or a combination thereof for processing the optical data received at the light detectors and continuously determining the heart rate based on the optical data. The optical data may be combined with data from one or more motion sensors, e.g., accelerometers and/or gyroscopes, to minimize or eliminate noise in the heart rate signal caused by motion or other artifacts (or with other optical data of another wavelength).

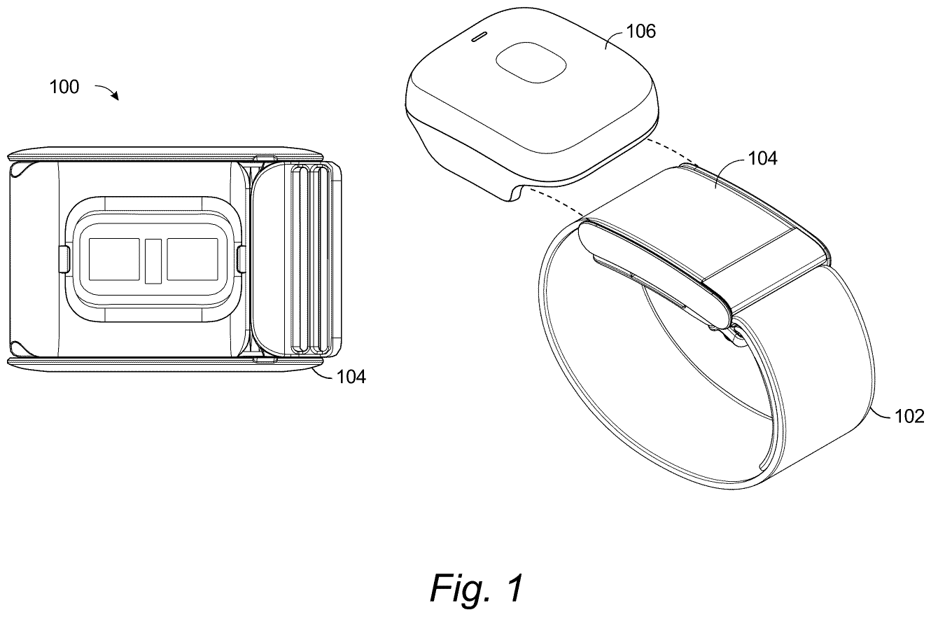

FIG. 1 shows a physiological monitoring device. The overall system 100 may include a device 104 (which may or may not include a display screen or other user interface) generally configured for physiological monitoring. The system 100 may further include a removable and replaceable battery 106 for recharging the device 104. A strap 102 may be provided, and may include any arrangement suitable for retaining the device 104 in a position on a wearer's body for acquisition of physiological data as described herein. For example, the strap 102 may include slim elastic band formed of any suitable elastic material, for example, a rubber, a woven polymer fiber such as a woven polyester, polypropylene, nylon, spandex, and so forth. The strap 102 may be adjustable to accommodate different wrist sizes, and may include any latches, hasps, or the like to secure the device 104 in an intended position for monitoring a physiological signal. While a wrist-worn device is depicted, it will be understood that the device 104 may be configured for positioning in any suitable location on a user's body, based on the sensing modality and the nature of the signal to be acquired. For example, the device 104 may be configured for use on a wrist, an ankle, a bicep, a chest, or any other suitable location(s), and the strap 102 may be, or may include, a waistband or other elastic band or the like within an article of clothing or accessory. The device 104 may also or instead be structurally configured for placement on or within a garment, e.g., permanently or in a removable and replaceable manner. To that end, the device 104 may be structurally configured for placement within a pocket, slot, and/or other housing that is coupled to or embedded within a garment. In such configurations, the garment may include sensing windows or other pathways such that the device 104 can sense physiological and/or biomechanical parameters from a user wearing a garment that includes the device 104 therein or thereon.

The system 100 may include any hardware components, subsystems, and the like to provide various functions such as data collection, processing, display, and communications with external resources. For example, the system 100 may include a heart rate monitor using, e.g., photoplethysmography, electrocardiogram any other technique(s). The system 100 may be configured such that, when placed for use about a wrist, the system 100 initiates acquisition of physiological data from the wearer. In some embodiments, the pulse or heart rate may be taken using an optical sensor coupled with one or more light emitting diodes (LEDs), all directly in contact with the user's wrist. The LEDs may be positioned to direct illumination toward the user's skin, and may be accompanied by one or more photodiodes or other photodetectors suitable for measuring illumination from the LEDs that is reflected and/or transmitted by the wearer's skin.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure, and the like, as well environmental or contextual parameters such as ambient light, ambient temperature, humidity, time of day, and the like. The system 100 may also include other sensors such as accelerometers and/or gyroscopes for motion detection, and sensors for environmental temperature sensing, electrodermal activity (EDA) sensing, galvanic skin response (GSR) sensing, and the like.

The system 100 may include one or more sources of battery life, such as a first battery environmentally sealed within the device 104 and a battery 106 that is removable and replaceable to recharge the battery in the device 104. The system 100 may perform numerous functions related to continuous monitoring, such as automatically detecting when the user is asleep, awake, exercising, and so forth, and such detections may be performed locally at the device 104 or at a remote service coupled in a communicating relationship with the device 104 and receiving data therefrom. In general, the system 100 may support continuous, independent monitoring of a physiological signal such as a heart rate, and acquired data may be stored on the device 104 until it can be uploaded to a remote processing resource for more computationally expensive analysis.

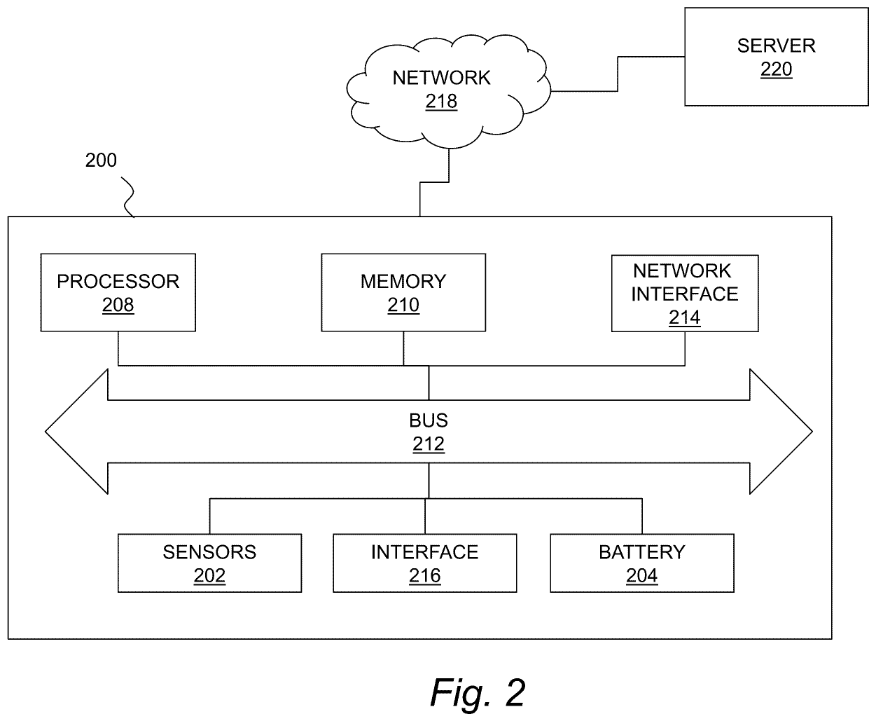

FIG. 2 is a block diagram of an exemplary computing device 200 that may be used in to perform any of the methods provided by exemplary embodiments. The computing device may, for example, be a device used for continuous physiological monitoring. The device may also or instead be any of the local computing devices described herein, such as a desktop computer, laptop computer, smart phone. The device may also or instead be any of the remote computing resources described herein, such as a web server, a cloud database, a file server, an application server, or any other remote resource or the like. While described as a physical device, it will be understood that the exemplary computing device 200 may also or instead be realized as a virtual computing device such as a virtual machine executing a web server or other remote resource in a cloud computing platform. In general, the device 200 may include one or more sensors 202, a battery 204, storage 206, a processor 208, memory 210, a network interface 214, and a user interface 216, or virtual instances of one or more of the foregoing.

The sensors 202 may include any sensor or combination of sensors suitable for heart rate monitoring as contemplated herein, as well as sensors 202 for detecting calorie burn, position (e.g., through a Global Positioning System or the like), motion, activity and so forth. In one aspect, this may include optical sensing systems including LEDs or other light sources, along with photodiodes or other light sensors, that can be used in combination for photoplethysmography measurements of heart rate, pulse oximetry measurements, and other physiological monitoring.

The sensors 202 may also or instead include one or more sensors for activity measurement. In some embodiments, the system may include one or more multi-axes accelerometers and/or gyroscope to provide a measurement of activity. In some embodiments, the accelerometer may further be used to filter a signal from the optical sensor for measuring heart rate and to provide a more accurate measurement of the heart rate. In some embodiments, the wearable system may include a multi-axis accelerometer to measure motion and calculate distance. Motion sensors may be used, for example, to classify or categorize activity, such as walking, running, performing another sport, standing, sitting or lying down. The sensors 202 may, for example, include a thermometer for monitoring the user's body or skin temperature. In one embodiment, the sensors 202 may be used to recognize sleep based on a temperature drop, Galvanic Skin Response data, lack of movement or activity according to data collected by the accelerometer, reduced heart rate as measured by the heart rate monitor, and so forth. The body temperature, in conjunction with heart rate monitoring and motion, may be used, e.g., to interpret whether a user is sleeping or just resting, as well as how well an individual is sleeping. The body temperature, motion, and other sensed data may also be used to determine whether the user is exercising, and to categorize and/or analyze activities as described in greater detail below. In another aspect, the sensors 202 may include one or more contact sensors, such as a capacitive touch sensor or resistive touch sensor, for detecting placement of a physiological monitor for use on a user. More generally, the sensors 202 may include any sensor or combination of sensors suitable for monitoring geographic location, physiological state, exertion, movement, and so forth in any manner useful for physiological monitoring as contemplated herein.

The battery 204 may include one or more batteries configured to allow continuous wear and usage of the wearable system. In one embodiment, the wearable system may include two or more batteries, such as a removable battery that may be removed and recharged using a charger, along with an integral battery that maintains operation of the device 200 while the main battery charges. In another aspect, the battery 204 may include a wireless rechargeable battery that can be recharged using a short range or long range wireless recharging system.

The processor 208 may include any microprocessor, microcontroller, signal processor or other processor or combination of processors and other processing circuitry suitable for performing the processing steps described herein. In general, the processor 208 may be configured by computer executable code stored in the memory 210 to provide activity recognition and other physiological monitoring functions described herein.

In general the memory 210 may include one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, optical disks, USB flash drives), and the like. In one aspect, the memory 210 may include a computer system memory or random access memory, such as DRAM, SRAM, EDO RAM, and the like. The memory 210 may include other types of memory as well, or combinations thereof, as well as virtual instances of memory, e.g., where the device is a virtual device. In general, the memory 210 may store computer readable and computer-executable instructions or software for implementing methods and systems described herein. The memory 210 may also or instead store physiological data, user data, or other data useful for operation of a physiological monitor or other device described herein, such as data collected by sensors 202 during operation of the device 200.

The network interface 214 may be configured to wirelessly communicate data to a server 220, e.g., through an external network 218 such as any public network, private network, or other data network described herein, or any combination of the foregoing including, e.g., local area networks, the Internet, cellular data networks, and so forth. Where the device is a physiological monitoring device, the network interface 214 may be used, e.g., to transmit raw or processed sensor data stored on the device 200 to the server 220, as well as to receive updates, receive configuration information, and otherwise communicate with remote resources and the user to support operation of the device. More generally, the network interface 214 may include any interface configured to connect with one or more networks, for example, a Local Area Network (LAN), a Wide Area Network (WAN), the Internet, or a cellular data network through a variety of connections including, but not limited to, standard telephone lines, LAN or WAN links (for example, 202.11, T1, T3, 56 kb, X.25), broadband connections (for example, ISDN, Frame Relay, ATM), wireless connections, or some combination of any or all of the above. The network interface 212 may include a built-in network adapter, network interface card, PCMCIA network card, card bus network adapter, wireless network adapter, USB network adapter, modem or any other device suitable for interfacing the computing device 200 to any type of network capable of communication and performing the operations described herein.

The user interface 216 may include any components suitable for supporting interaction with a user. This may, for example, include a keypad, display, buzzer, speaker, light emitting diodes, and any other components for receiving input from, or providing output to, a user. In one aspect, the device 200 may be configured to receive tactile input, such as by responding to sequences of taps on a surface of the device to change operating states, display information and so forth. The user interface 216 may also or instead include a graphical user interface rendered on a display for graphical user interaction with programs executing on the processor 208 and other content rendered by a physical display of device 200.

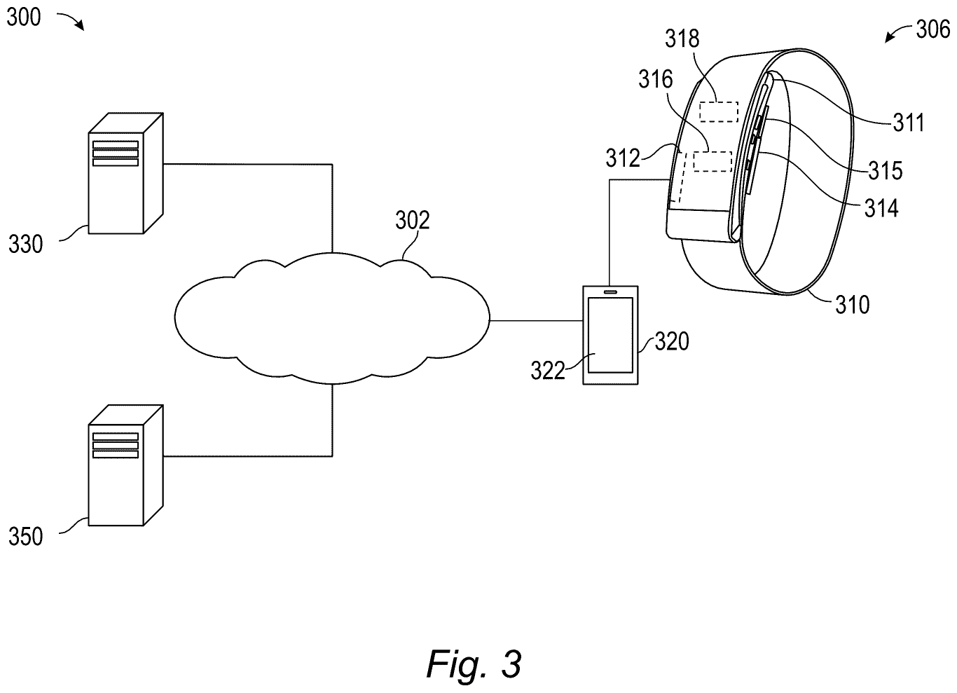

FIG. 3 illustrates a physiological monitoring system. More specifically, FIG. 3 illustrates a system 300 facilitating physiological monitoring that may be used with any of the methods or devices described herein. In general, the system 300 may include a physiological monitor 306, a user device 320, a remote server 330 with a remote data processing resource (such as any of the processors or processing resources described herein), and one or more other resources 350, all of which may be interconnected through a data network 302.

The data network 302 may be any of the data networks described herein. For example, the data network 302 may be any network(s) or internetwork(s) suitable for communicating data and information among participants in the system 300. This may include public networks such as the Internet, private networks, telecommunications networks such as the Public Switched Telephone Network or cellular networks using third generation (e.g., 3G or IMT-2000), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 300. This may also include local or short range communications networks suitable, e.g., for coupling the physiological monitor 306 to the user device 320, or otherwise communicating with local resources.

The physiological monitor 306 may, in general, be any physiological monitoring device, such as any of the wearable monitors or other monitoring devices described herein, such as bracelet 100 in FIG. 1. Thus, the physiological monitor 306 may generally be shaped and sized to be worn on a wrist or other appendage of a user and retained in a desired orientation relative to the appendage with a strap 310 or other attachment mechanism. The physiological monitor 306 may include a wearable housing 311, a network interface 312, one or more sensors 314, one or more light sources 315, a processor 316, a memory 318, and a wearable strap 310 for retaining the physiological monitor 306 in a desired location on a user.

In general, the physiological monitor 306 may include a wearable physiological monitor configured to acquire heart rate data and/or other physiological data from a wearer. More specifically, the wearable housing 311 of the physiological monitor 306 may be configured such that a user can wear a wearable physiological monitor 306 to acquire heart rate data and/or other physiological data from the user in a substantially continuous manner. The wearable housing 311 may be configured for cooperation with a strap 310 or the like, e.g., for engagement with an appendage of a user.

The network interface 312 may be configured to coupled one or more participants of the system 300 in a communicating relationship, e.g., with the remote server 330. The network interface 312 may be configured to couple one or more participants of the system 300 in a communicating relationship, e.g., with the remote resource using techniques such as Bluetooth, Wi-Fi (Wireless-Fidelity), the mobile network (3G, 4G, 5G, . . . ), or near field communication (NFC).

The one or more sensors 314 may include any of the sensors described herein, or any other sensors suitable for physiological monitoring. By way of example and not limitation, the one or more sensors 314 may include one or more of a light source, and optical sensor, an accelerometer, a gyroscope, a temperature sensor, a galvanic skin response sensor, an environmental sensor (e.g., for measuring ambient temperature, humidity, lighting, and the like), a geolocation sensor, a temporal sensor, an electrodermal activity sensor, and the like. The one or more sensors 314 may be disposed in the wearable housing 311, or otherwise positioned and configured for capture of data for physiological monitoring of a user. In one aspect, the one or more sensors 314 may include a light detector configured to provide data to the processor 316 for calculating a heart rate variability. The one or more sensors 314 may also or instead include an accelerometer configured to provide data to the processor 316, e.g., for detecting a sleep state, a waking event, exercise, and/or other user activity. In an implementation, the one or more sensors 314 may measure a galvanic skin response of the user.

The processor 316 and memory 318 may be any of the processors and memories described herein, and may be suitable for deployment in a physiological monitoring device. In one aspect, the memory 318 may store physiological data obtained by monitoring a user with the one or more sensors 314. The processor 316 may be configured to obtain heart rate data from the user based on the data from the sensors 314. The processor 316 may be further configured to assist in a determination of a condition of the user, such as whether the user has an infection or other condition of interest as described herein.

The one or more light sources 315 may be coupled to the wearable housing 311 and controlled by the processor 316. At least one of the light sources 315 may be directed toward the skin of a user's appendage. Light from the light source 315 may be detected by the one or more sensors 314.

The system 300 may further include a remote data processing resource executing on a remote server 330. The remote data processing resource may be any of the processors described herein, and may be configured to receive data transmitted from the memory 318 of the physiological monitor 306, and to evaluate a condition of the user such as whether the user has an infection or other condition of interest as described herein.

The system 300 may also include one or more user devices 320, which may work together with the physiological monitor 306, e.g., to provide a display for user data and analysis, and/or to provide a communications bridge from the network interface 312 of the physiological monitor 306 to the data network 302 and the remote server 330. For example, the physiological monitor 306 may communicate locally with the user device 320, such as a smartphone of a user, via short-range communications, e.g., Bluetooth, or the like, e.g., for the exchange of data between the physiological monitor 306 and the user device 320, and the user device 320 may communicate with the remote server 330 via the data network 302. Computationally intensive processing may be performed at the remote server 330, which may have greater memory capabilities and processing power than the physiological monitor 306 that acquires the data. However, it will be understood that processing may also or instead be performed at one or more of the physiological monitor 306, the user device 320, and so on. That is, it will be understood that one or more of the steps related to techniques for physiological monitoring as described herein, or sub-steps, calculations, functions, and the like related thereto, can be performed locally, remotely, or some combination of these. For example, these steps may be performed locally on a wearable device, remotely on a server or other remote resource, on an intermediate device such as a local computer used by the user to access the remote resource, or any combination of these.

The user device 320 may include any computing device as described herein, including without limitation a smartphone, a desktop computer, a laptop computer, a network computer, a tablet, a mobile device, a portable digital assistant, a cellular phone, a portable media or entertainment device, and so on. The user device 320 may provide a user interface 322 for access to data and analysis by a user, and/or to control operation of the physiological monitor 306. The user interface 322 may be maintained by an application executing locally on the user device 320, or the user interface 322 may be remotely served and presented on the user device 320, e.g., from the remote server 330 or the one or more other resources 350.

In general, the remote server 330 may include data storage, a network interface, and/or other processing circuitry. The remote server 330 may process data from the physiological monitor 306, and the remote server 330 may perform any of the analyses described herein, and may host a user interface for remote access to this data, e.g., from the user device 320. The remote server 330 may include a web server or other programmatic front end that facilitates web-based access by the user devices 320 and/or the physiological monitor 306 to the capabilities of the remote server 330 or other components of the system 300.

The other resources 350 may include any resources that can be usefully employed in the devices, systems, and methods as described herein. For example, these other resources 350 may include without limitation other data networks, human actors (e.g., programmers, researchers, annotators, editors, analysts, and so forth), sensors (e.g., audio or visual sensors), data mining tools, computational tools, data monitoring tools, algorithms, and so forth. The other resources 350 may also or instead include any other software or hardware resources that may be usefully employed in the networked applications as contemplated herein. For example, the other resources 350 may include payment processing servers or platforms used to authorize payment for access, content, or option/feature purchases, or otherwise. In another aspect, the other resources 350 may include certificate servers or other security resources for third-party verification of identity, encryption or decryption of data, and so forth. In another aspect, the other resources 350 may include a desktop computer or the like co-located (e.g., on the same local area network with, or directly coupled to through a serial or USB cable) with a user device 320, physiological monitor 306, and/or remote server 330. In this case, the other resources 350 may provide supplemental functions for other components of the system 300.

The other resources 350 may also or instead include one or more web servers that provide web-based access to and from any of the other participants in the system 300. While depicted as a separate network entity, it will be readily appreciated that the other resources 350 (e.g., a web server) may also or instead be logically and/or physically associated with one of the other devices described herein, and may for example, include or provide a user interface 322 for web access to a remote server 330 or a database in a manner that permits user interaction through the data network 302, e.g., from the physiological monitor 306 and/or the user device 320.

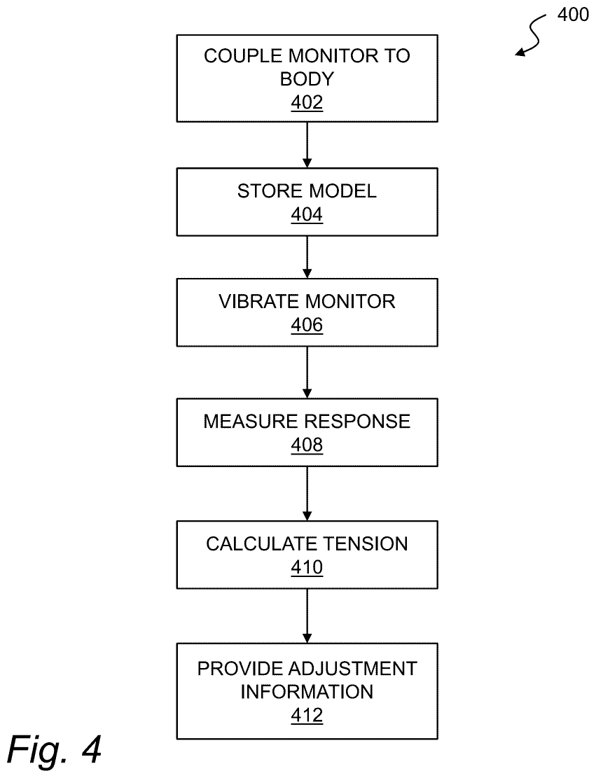

FIG. 4 is a flow chart illustrating a method 400 for measuring tightness of a wearable monitor and providing actionable feedback to a user based on a physical model. The tightness of a wearable monitor can have an impact on performance. For example, when an optical monitor, such as a photoplethysmography monitor or blood oxygenation monitor, is too loose, the resulting signal can deteriorate due to poor optical coupling between the sensor and the skin. To help ensure an optimal level of tightness, a physical model such as a spring model or a resonance model may be created to characterize movement of the wearable monitor when elastically retained in tension about a body part. The wearable monitor may then be vibrated, and a measured response to these vibrations may be used with the physical model to infer the tension, e.g., by calculating a tension for the physical model that yields a response to the vibration equivalent to the measured response. The inferred tension may then be used to provide adjustment information to the user, e.g., to tighten, loosen, reposition, and/or otherwise adjust the monitor for improved or proper operation.

As shown in step 402, the method 400 may include coupling a wearable monitor to a body of a user. The monitor may include a physiological monitor, an optical monitor, a photoplethysmography system, a pulse oxygen monitor, or any of the other wearable physiological monitors described herein, or any other monitor that might be coupled to a body of a user with an elastic strap, band, fabric, elastic clothing, or the like. For example, the monitor may be coupled to a wrist of a user with a wristband. The monitor may instead be coupled to a chest, a bicep, an ankle, a calf, a torso, a waist, a leg, an arm, or some other body part with an elastic strap or an elastic article of clothing formed of an athletic knit such as Lycra, spandex, elastane, one or more elastic straps, or some other fabric or elastic material formed of a polymer, polyurethane rubber, or the like. The monitor may usefully include a haptic output device and motion sensors such as accelerometers, gyroscopes, and/or magnetometers in order to provide a stimulus and response for fit detection as described herein. While the techniques described herein are generally described in the context of wearable physiological monitors, the techniques may more generally be applied to any system where proper performance depends on a tension (or corresponding normal force) with which a device is elastically retained in an intended position, and all such uses are intended to fall within the scope of this disclosure unless expressly stated otherwise.

As shown in step 404, the method 400 may include storing a model for physical behavior of the wearable monitor to motion. This may, for example, include a physical model such as a resonance model characterizing how the wearable monitor and any elastic tensioning members move in response to an applied force, e.g., as a function of tension in one or more elastic tensioning members (or a lumped characterization of same). The model may be any empirical, analytical, or other model suitable for relating a vibration response to a tension in the elastic tensioning members. In one aspect, a resonance model provides a useful approximation that has been demonstrated to yield accurate tension calculations suitable for the purposes contemplated herein. One such resonance model based on a spring system is now discussed in greater detail by way of example. However, the physical model may more generally include any suitable type of system model based on mechanical inputs and resulting motion (or optical response, as further discussed below). The model of physical behavior may also or instead include an empirical or data driven model trained to identify tension based on training data sets of mechanical/optical responses labeled by a suitable training metric such as physical tension, device fit, measurement accuracy, and so forth.

In general, the tightness of the wearable sensor may be characterized as the pressure that the sensor optical interface applies to the skin to maintain contact. Given the total normal force pushing the strap to the skin (F) and the contact area of a sensor (A), assuming the pressure is uniformly distributed, the strap tightness may be calculated as F/A. The uniform distribution of the pressure over the contract area is a very strong assumption, especially during motion. The force F between the sensor and the skin when the sensor is facing up and when the second facing down may be adjusted according to the forces of gravity as follows:

$$F = sin ( α ) 2 f + mg \\ and \\ F = cos ( α ) 2 f - mg$$

respectively, where α is the angle between the strap and the garment, f is the tightness of the garment, m is the weight of the sensor, and g is the gravity coefficient (acceleration due to gravity). When the garment, strap, or other elastic tensioning member is elastic with a spring constant of k:

f=k(dx)

where dx is the change of the strap length and f is the tightness of the elastic tensioning member. In practice, k is a monotonic function of dx over the elastic range of interest. Given these equations, a direct relationship can be derived between tightness and the force between strap and skin in a stationary state. The force between the skin and the sensor in motion can be calculated given an acceleration vector and the weight of the sensor. This framework generally confirms that physical displacement of the device is a function of strap tension and applied forces, and that if k is known or calculated, the tightness (and consequently the force between the sensor and the skin) can also be calculated based on an acceleration vector and a mass of the device. However, directly calculating tension on this basis requires at least calibration of the mechanical force applied by a stimulus (e.g., a haptic device) in response to a control signal. Thus, a resonance model may also or instead be advantageously employed to infer a spring constant based on resonant response to a frequency sweep or the like.

When a force is placed on a material, the material stretches or compresses in response to the force. The force per unit area is the stress (σ). The extent of the stretching/compression produced as the material responds to stress is the strain (ϵ). Strain is measured by the ratio of the difference in length ΔL, L to original length L₀, along the direction of the stress, i.e., ϵ=ΔL/L₀.

Resonance describes the phenomenon of increased amplitude that occurs when the frequency of an applied force is equal or close to a natural frequency of the system on which it acts. When an oscillation force is applied at a resonant frequency of a dynamic system, the system will oscillate at a higher amplitude than when the same force is applied at other, non-resonant frequencies. The quality factor relates the maximum or peak energy stored in the circuit (the reactance) to the energy dissipated (the resistance) during each cycle of oscillation meaning that it is a ratio of resonant frequency to bandwidth and the higher the circuit Q, the smaller the bandwidth, Q=fᵣ/BW.

These properties may be used as described herein to characterize a frequency response of the sensor/elastic combination to a mechanical stimulus such as vibration of a haptic output element. A model based on these properties may be stored in any suitable location, e.g., on a memory of the wearable monitor, on a memory of a personal computing device or the like used to perform tension calculations, or on a remote server that performs the tension calculations and provides actionable feedback to the user through the personal computing device (or any combination of these). In one aspect, the resonance model may include an analytical model characterizing strap tension, e.g., of a wrist worn device, as a function of the resonant frequency of the spring system. Depending on the desired range and accuracy of the calculation, this may be a linear model, an exponential model, a quadratic model, or any other model that physically describes the spring system, and that can be fit to experimental data for the spring system. In another aspect, the resonance model may be an empirical or experimental model correlating, e.g., measured resonance frequencies to measured strap tensions. In another aspect, particularly where the observed response does not yield to simple mathematical models, the experimental data may be modeled as a lookup table or the like where tension can be looked up (or interpolated) based on a measured resonant frequency. The actual resonance may be estimated, e.g., based on the wavelength that maximizes measured accelerometer response to haptic input (e.g., a ratio of accelerometer signal to haptic input signal, each of which may be measured in the frequency domain, e.g., to reduce the effects of phase changes or other artifacts).

As shown in step 406, the method 400 may include vibrating the wearable monitor. The vibration may occur upon user request or automatically during a specified event or time. For example, the device may include a button, such as a physical button on the device, or a button in a user interface of another device, that a wearer can press to check for proper fit of a device. In another example, the device may automatically test for fit in response to detected events, such as detecting that a user has put the device on, or detecting a deterioration in data quality below a predetermined threshold while the device is being worn. Vibrating the wearable monitor may, for example, include causing a vibration of the wearable monitor by activating a haptic output element, piezo element, buzzer, eccentric motor, linear vibration motor or other linear haptic actuator, or other vibrator or the like associated with (e.g., mechanically coupled to and/or within a housing of) the wearable monitor. This may include a rotary haptic element, a linear haptic element, or any other haptic element. While rotary vectors for vibration may complicate individual spring measurements, the location of a resonant response can advantageously be performed without resolving rectilinear components of the haptic output and without calibrating haptic amplitude. The control signal for the vibration may, for example, include a chirp signal that increases or decreases in frequency with the passage of time in order to sweep a range of frequencies to locate a resonant frequency (or range of resonant frequencies) for the wearable monitor and elastic tensioning member(s). It will be understood, however, that other signals are also or instead possible for use herein, such as any signal that covers a sufficiently large frequency range for location of resonance. In one aspect, a signal such as a swept sine or cosine may be employed, for example:

$$y = sin [ 2 π ( at + f ) t ]$$

More generally, any linear-frequency chirp, exponential chirp, hyperbolic chirp, or other function that increases or decreases a signal frequency over time may be used. The frequency sweep may be continued until the earlier of (1) achieving a predetermined confidence level for a resonance detection (or a corresponding tension calculation) or (2) a testing timeout. Thus, for example, if a reliable tension measurement cannot be obtained in one-hundred eight seconds (or some other window of time suitable for one or more complete sweeps of a target frequency range) the test may be terminated and an error message may be provided.

As shown in step 408, the method 400 may include measuring a response of the wearable monitor to the vibration, such as by measuring the response with one or more gyroscopes, accelerometers, optical sensors (e.g., by measuring movement against the skin of the user), or any combination of the foregoing or the like. This data may be processed by the wearable monitor or transmitted to a remote resource such as a personal computing device of the user or a remote server for analysis and determination of the elastic tension or circumferential forces retaining the wearable monitor in place.

As shown in step 410, the method 400 may include calculating a tension for the elastic tensioning member (e.g., clothing, strap, band, or the like) that retains the wearable monitor on the body. In general, this may include locating a resonant frequency of the strap/monitor system in response to the chirp or other stimulus, and using this resonant frequency to calculate the spring constant of the system and infer the radial tension. In general, the resonant frequency will be identified at a frequency corresponding to a maximum amplitude in the accompanying mechanical response. Where an analytical model is derived and employed, the tension (or other suitable metric) may be calculated by inputting the measured resonant frequency into the analytically derived equation to calculate tension. As noted above, the model may also or instead be an experimental or empirical model that correlates resonance to tension based on experimental observations. The experimental model may be embodied, e.g., in a look up table, a linear regression model, or some other model that fits measured resonance data to measured strap tension data in a statistically significant manner. Where a lookup table is used, interpolation (e.g., linear interpolation) may also be used if/as appropriate to evaluate tension for interstitial frequencies between values stored in the lookup table. In the latter case, measuring tightness can be performed by simply stimulating the device with a frequency sweep, locating a peak in resonant response, and then, given this resonant frequency, either looking up the tension in a lookup table, or calculating the tension using the regression model or the like.

As shown in step 412, the method 400 may include providing adjustment information to the user, e.g., using any of the techniques described herein. This may, for example, include a quantitative statement of tension, e.g., a circumferential or normal force determined by the calculation, expressed in Newtons or some other physical units. This may also or instead include a score, e.g., of −10 to 10, with zero being the optimal tension, scores between −5 and 5 being acceptable for accurate data acquisition, and anything outside the range of −10 to 10 unlikely to yield accurate or meaningful data. In another aspect, the adjustment information may include qualitative assessments of whether the current tension is within an acceptable range, such as “too tight” (e.g., corresponding to a score as described above greater than 5), “too loose” (e.g., corresponding to a score less than −5), “okay” (e.g., corresponding to a score between −5 and 5), or “optimal” (e.g., corresponding to a score between −1 and 1), or using any similar range bound natural language descriptions. Where information is available concerning the circumference and/or material of the elastic tensioning member(s), or where the model otherwise provides suitable output or analysis, this may include actionable instructions such as “tighten strap at least one millimeter.” In one aspect, the actionable instructions may include a visual component illustrating the instructions. Also or instead, if a strap has a built-in, controllable tensioning system that provides specific feedback (e.g., audio, visual, or tactile feedback), actionable instructions may include specific instructions such as “tighten strap three clicks,” or the like. In another aspect, if the strap has a built-in, automatic tension controller, the method 400 may include generating control signals to automatically adjust the tension of the strap toward a predetermined tension target.

The adjustment information may be displayed on the wearable monitor or on a local computing device. In one aspect, the adjustment information may be displayed concurrently with one or more other quantitative or qualitative pieces of information such as current physiological measurements for the user. If the wearable monitor detects a user adjustment to strap tension, the monitor may automatically re-test strap tension and/or update the tension metrics or recommendations.

It will be understood that the tension measurement may usefully be repeated under a variety of conditions. For example, the tension measurement may initially be performed when a wearable monitor is placed on the body. The tension measurement may be repeated on a regular schedule, e.g., as a maintenance function, or under conditions indicating a change in tension such as a deteriorating signal strength or decreasing quality/confidence for a physiological metric such as heart rate. In one aspect, the tension measurement may be repeated continuously for a period of time, e.g., at regular, short intervals, when available information indicates that the device is being worn and the tension is outside an acceptable range. In this case, the tension measurement may be repeated until the tension is determined to be in the acceptable range or until a timeout limit is reached. In the latter case, an error notification may be reported to the user, along with a warning that accurate data is not currently being acquired. The tension measurement, or other evaluation of fit, may also or instead be performed on-demand, based on a predetermined user interaction such as touching a button on a user interface, double tapping the device, or the like.

According to the foregoing, a system described herein includes a wearable monitor and a remote processing resource, which may be a remote server or a personal computing device such as a laptop or smart phone for a user of the wearable monitor. The wearable monitor may include a processor, a sensor, and a haptic output element. Computer executable code stored in a memory of the wearable monitor may configure the processor to cause a vibration of the haptic output element and receive a response to the vibration from the sensor. The remote processing resource may be coupled in a communicating relationship with the wearable monitor, and may include a second memory storing a physical model of the wearable monitor and a second processor configured to receive the response to the vibration from the wearable monitor, to apply the physical model to calculate a tension of the wearable monitor about a body part of a user, and to communicate tension information to the user based on the tension. As described herein, the tension may be reported as a physical measurement, an objective fit score, a human-readable evaluation, an instruction for adjustments, or some combination of these.

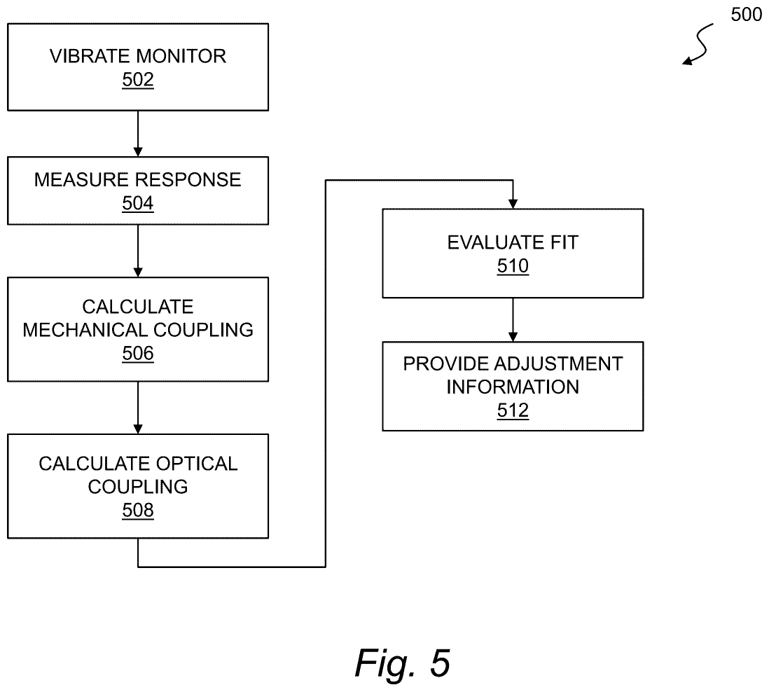

FIG. 5 is a flow chart illustrating a method 500 for measuring fit of a wearable monitor. In general, mechanical and optical coupling of the wearable monitor can be measured based on a low resolution haptic stimulus, and used to evaluate fit and provide actionable feedback. As a significant advantage, this approach mitigates the need to calibrate haptic output, or to generate time varying control signals such as frequency sweeps. Instead, the method 500 can be deployed with, e.g., a binary haptic device that is only operable in an ‘on’ and ‘off’ mode, and/or that has an unknown and/or varying mechanical orientation relative to the device being tested.

As shown in step 502, the method 500 may include causing a vibration of a wearable monitor coupled to a body of a user. The monitor may include a physiological monitor, an optical monitor, a photoplethysmography system, a pulse oxygen monitor, or any of the other wearable physiological monitors described herein that might be coupled to a body of a user with an elastic strap, band, fabric, or the like. For example, the monitor may be coupled to a wrist of a user with a wristband. The monitor may instead be coupled to a chest, a bicep, an ankle, a calf, a torso, a waist, a leg, an arm, or some other body part with an elastic strap or an elastic article of clothing formed of an athletic knit such as Lycra, spandex, elastane, or some other fabric formed of a polymer, polyurethane rubber, or the like, or any of the other elastic straps or the like described herein. While the techniques described herein are generally described in the context of wearable physiological monitors, the techniques may more generally be applied to any system where proper performance depends on a tension with which a monitor or sensor is elastically retained in an intended position, and all such uses are intended to fall within the scope of this disclosure unless expressly stated otherwise.

The vibration of the wearable monitor may occur upon user request or automatically during a specified event or time, such as when the user puts on the wearable monitor, or more generally at any times and/or using any user interactions described herein. Vibrating the wearable monitor may, for example, include causing a vibration of the wearable monitor by activating a haptic output element such as a piezo element, buzzer, acentric motor, or other vibrator or the like associated with (e.g., mechanically coupled to and/or within a housing of) the wearable monitor. In some embodiments, the haptic output element may be a linear haptic output element configured to deliver haptic outputs along a particular axis. In some embodiments, the vibration may last for one minute or longer.

As shown in step 504, the method 500 may include measuring a response of the wearable monitor to the vibration. For example, measuring the response may include receiving motion data during the vibration, such as data from one or more gyroscopes, accelerometers, or the like, or combinations of the foregoing. Measuring the response may also or instead include receiving optical data during the vibration from one or more light detectors. The response may be processed by the wearable monitor or transmitted to a remote resource such as a personal computing device of the user or a remote server for analysis of the clastic tension or circumferential forces retaining the wearable monitor in place.

As shown in step 506, the method 500 may include calculating a level of mechanical coupling of the wearable monitor about the body based on the response. In general, this includes the coupling between movement along two or more axes. For example, the level of mechanical coupling between a first axis and a second axis may be measured as a phase relationship between a force along the first axis and a force along the second axis. In general, the tighter the wearable monitor is about the body, the smaller the phase relationship (e.g., the closer the response) and thus the larger the mechanical coupling. For a number of axial sensors, the couplings between forces and motions in each axis pair can be inferred from the cross-correlation between measured motion for each axis of that axis pair over time, which in this context, measures the correlation among movements in each axis over time. Thus, for example, a three axis accelerometer system will yield three cross correlations in XY, XZ, and YZ. Similarly, gyroscopic data may yield three cross correlations in rotation for three similar axis pairs. While instantaneous measurements may not provide meaningful results in this context, the average for each axis pair for large numbers of samples will tend to converge on a true cross-correlation for that axis pair where there is actual mechanical coupling between the axes. As such, data may be acquired over an extended interval such as thirty seconds, sixty seconds, ninety seconds, or one hundred eight seconds and/or until a level of confidence in the calculated value(s) meets a predetermined threshold. In this context, the predetermined threshold may be a statistical measure of confidence based on, e.g., a variance in calculated results or mean square error relative to a measurement benchmark.

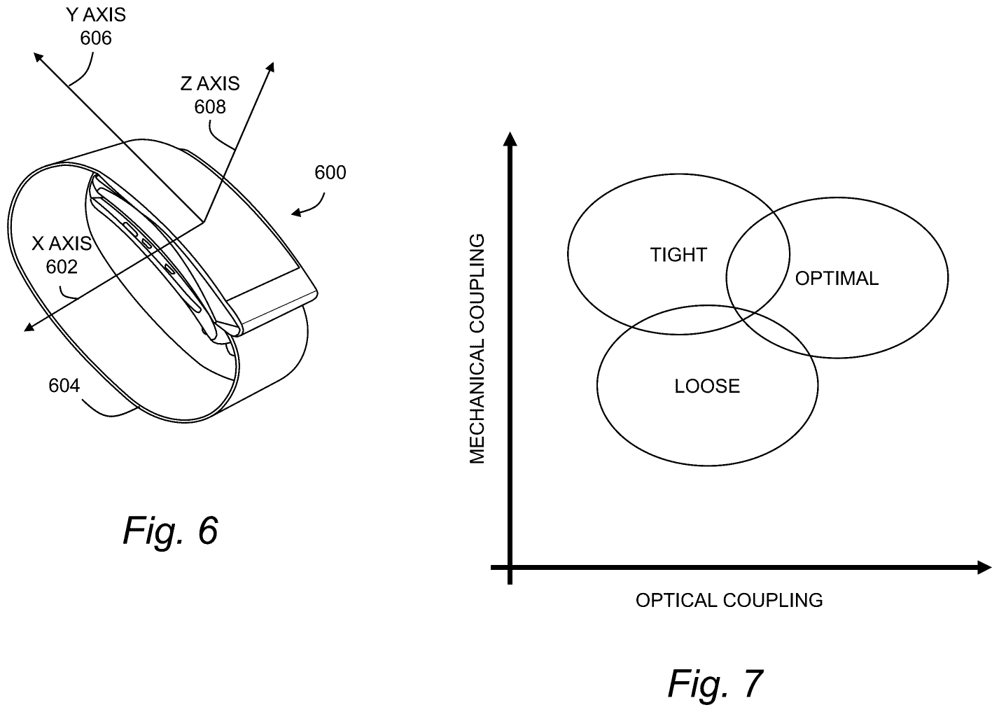

While any of the foregoing cross-correlations may be used to measure mechanical coupling as contemplated herein, and each is generally correlated to tightness, there has been observed a monotonic relationship between the ZY mechanical coupling and strap tightness, where the Z axis is normal to the skin, and the Y axis is parallel to the skin and parallel to the strap (as illustrated in FIG. 6 below). This mechanical coupling may be used to estimate strap tension based exclusively on the ZY mechanical coupling. Other couplings between accelerometer and/or gyroscopic axes may also or instead be used, for example, by themselves, in combination with ZY coupling, or as a supplemental or quality control check on inferences based on the ZY coupling.

As shown in step 508, the method 500 may include calculating a level of optical coupling of the wearable monitor about the body based on the response. The level of optical coupling may be calculated independently from the level of mechanical coupling based on the motion data and the optical data. In this case, a similar cross-correlation may be used, however with optical data correlated to acceleration in order to characterize a ratio between two components of the optical signal: heart rate signal (expected to be independent from instantaneous motion) and motion artifacts (expected to be dependent on measured, instantaneous motion). In some embodiments, the motion data may have at least three axes (i.e., from a three-axis IMU, gyroscope, accelerometer, or the like), with X axis data in particular demonstrated as highly correlated to strap tension. As illustrated in FIG. 6 below, the X axis of the device, in this context, is parallel to the skin and perpendicular to the strap.

As shown in step 510, the method 500 may include evaluating fit. This may include scaling, transforming, or otherwise processing the mechanical and optical coupling to obtain a conclusion concerning quantitative tension (e.g., a specific physical measure of tension) or qualitative tension (e.g., a category or human-readable evaluation of tension).

In one aspect, proper fit may be determined by applying ranges and/or thresholds to calculated mechanical and/or optical coupling. In some embodiments, the wearable monitor may be determined to be too tight if the level of mechanical coupling exceeds the threshold and the level of optical coupling is not within the range. In some embodiments, the wearable monitor may be determined to be too loose if the level of mechanical coupling does not exceed the threshold and the level of optical coupling is not within the range. In some embodiments, the wearable monitor may be determined to be at an acceptable tightness level and be coupled to an appendage of the body if the level of mechanical coupling exceeds the threshold and the level of optical coupling is within the range. It will be appreciated that numerical values are relatively arbitrary in this context, and will depend on the manner in which values for mechanical and optical coupling are calculated and reported. However, empirical ranges and thresholds may be readily established for discriminating between properly fitting and improperly fitting devices. It will also be understood that the conditions for proper fit of a strap such as a wrist strap or bicep strap may be different than the conditions for proper fit of a monitor in an article of clothing. Thus, for example, in some embodiments, the wearable monitor may be determined to be at an acceptable tightness level and be coupled to an article of clothing of the user if the level of mechanical coupling does not exceed the threshold and the level of optical coupling is within the range. The threshold and the range may be predetermined values based on the physical properties of wearable monitor, location of the device, physical properties of the tensioning member for the wearable monitor, targets for data quality, and so forth.

In one aspect, fit may be reported as a quantitative statement of tension, e.g., the circumferential or normal force determined by the calculation. In another aspect, the fit may be reported using a quantitative score such as a score on a range of −10 to 10, with zero being the optimal tension, scores between −5 and 5 being acceptable for accurate data acquisition, and anything outside the range of −10 to 10 unlikely to yield accurate or meaningful data. In another aspect, information about the fit may include qualitative assessments of whether the current tension is within an acceptable range, such as “too tight” (e.g., corresponding to a score as described above greater than 5), “too loose” (e.g., corresponding to a score less than −5), “okay” (e.g., corresponding to a score between −5 and 5), or “optimal” (e.g., corresponding to a score between −1 and 1), or using any similar range bound natural language descriptions. The adjustment information may also or instead include actionable instructions such as “tighten strap at least one millimeter,” where the physical adjustment estimate is calculated based on the position of the monitor and a corresponding estimate of the body circumference and/or material of the elastic tensioning member(s).

It should also be appreciated that, while various specific techniques are disclosed herein for measuring fit based on response to a haptic vibration or other mechanical stimulus—specifically location of a resonant frequency or measurement of mechanical/optical coupling—other techniques for measuring fit based on the response to haptic vibration may also or instead be used. In one aspect, two or more techniques (such as opto-mechanical coupling and a mathematical model using resonant frequency) may be used concurrently or sequentially, e.g., as a quality control measure or as an alternative where one technique does not yield a useful result.

As shown in step 512, the method 500 may include providing adjustment information, such as by displaying adjustment information to the user based on the level of mechanical coupling and the level of optical coupling. This may include communicating or displaying any of the fit information described herein to the user. In one aspect, this may include actionable instructions including, e.g., verbal or visual instructions concerning an adjustment. Also or instead, if a strap has a built-in, controllable tensioning system that provides specific feedback (e.g., audio, visual, or tactile feedback), actionable instructions may include specific instructions such as “tighten strap three clicks,” or similar. In another aspect, if the strap has a built-in, automatic tension controller, adjustment information may include generating control signals to automatically adjust the tension of the strap toward a predetermined tension target.

The adjustment information may be displayed on the wearable monitor, on a local computing device, or on any other suitable display device. In one aspect, the adjustment information may be displayed concurrently with one or more quantitative or qualitative pieces of information such as current physiological data for the user. If the wearable monitor detects a user adjustment to strap tension, the monitor may automatically re-test strap tension and/or update tension metrics or recommendations.

Adjustment information may be conditionally provided. For example, providing the adjustment information may be based on a threshold for the level of mechanical coupling and a range for the level of optical coupling. The adjustment information may include a determination of the location of the wearable monitor based on the threshold and the range, which may be reported to the user and/or applied to select a suitable model for evaluating fit as generally described herein.

It will be understood that the tightness measurement may usefully be repeated under a variety of conditions. For example, the tightness measurement may initially be performed when a wearable monitor is placed on the body. The tightness measurement may be repeated on a regular schedule, e.g., as a maintenance function, or under conditions indicating a change in tightness such as a deteriorating signal strength or decreasing quality/confidence for a physiological metric such as heart rate. In one aspect, the tightness measurement may be repeated continuously when adjustment information indicates that the tightness is outside an acceptable range. The tightness measurement may be repeated until the tightness is determined to be in the acceptable range or until a timeout limit has been reached.

According to the foregoing, a system described herein includes a wearable monitor and a remote processing resource, which may be a remote server or a personal computing device such as a laptop or smart phone for a user of the wearable monitor. The wearable monitor may include a processor, a sensor, and a haptic output element. Computer executable code stored in a memory of the wearable monitor may configure the processor to cause a vibration of the haptic output element and receive a response to the vibration from the sensor. The remote processing resource may be coupled in a communicating relationship with the wearable monitor, and may include a second memory storing a physical model of the wearable monitor and a second processor configured to receive the response to the vibration from the wearable monitor, to calculate a level of mechanical coupling of the wearable monitor about a body of a user based on the response, to calculate a level of optical coupling of the wearable monitor about the body independently from the level of mechanical coupling based on the response, and to communicate adjustment information to the user based on the level of mechanical coupling and the level of optical coupling.

FIG. 6 illustrates a coordinate system for measuring device motion. In general, the device 600 may be a wrist worn device or any of the other devices described herein. A coordinate system for the device 600 may include an x axis 602 in a plane substantially parallel to the user's skin where it contacts the device 600 when placed for use, but substantially normal to a strap 604 retaining the device in position. While it is understood that the strap is a complex contoured surface, in this context, normal to the strap should be understood to mean substantially normal to a plane intersecting a path following a band of tension circumferentially around the strap, or stated alternatively, substantially normal to a long axis of the device (e.g., the y axis 606 in FIG. 6) and substantially parallel to a short axis of the device (e.g., the x axis 602 in FIG. 6) as illustrated. The coordinate system may also include a y axis 606 substantially in the plane parallel to the user's skin and parallel to the strap 604, e.g., substantially parallel to the plane through the strap described above. The coordinate system may include a z axis 608 substantially perpendicular to a plane that is substantially parallel to the user's skin where it contacts the device 600 when placed for use. Accelerometers such as any of those described herein may be positioned to measure movement in each of the x axis 602, the y axis 606, and the z axis 608, e.g., for measurement of mechanical or optical coupling as described herein. Gyroscopes such as any of those described herein may also or instead be positioned to measure rotation about each of the x axis 602, the y axis 606, and the z axis 608, e.g., for measurement of mechanical or optical coupling as described herein.

FIG. 7 illustrates a mapping of optical and mechanical coupling to device fit. In general, ranges of mechanical coupling and optical coupling, as described herein, may be mapped to categories of fit. It will be understood that the categories and locations are conceptual only, and that the contours of any particular category, and/or the ranges of corresponding measured mechanical or optical coupling, will depend on the particular type of device, the location of the device, and the particular type of restraint system. Thus, for example, the nature of mechanical coupling and/or optical coupling for a properly tensioned device in a pocket of a wearable garment may be significantly different than the nature of mechanical coupling and/or optical coupling for a properly tensioned device strapped to a wearer's wrist. However, in general, ranges of optical and mechanical coupling have been demonstrated as reliably correlated to proper or improper tension, and may be used to generate user recommendations for adjustments as contemplated herein.

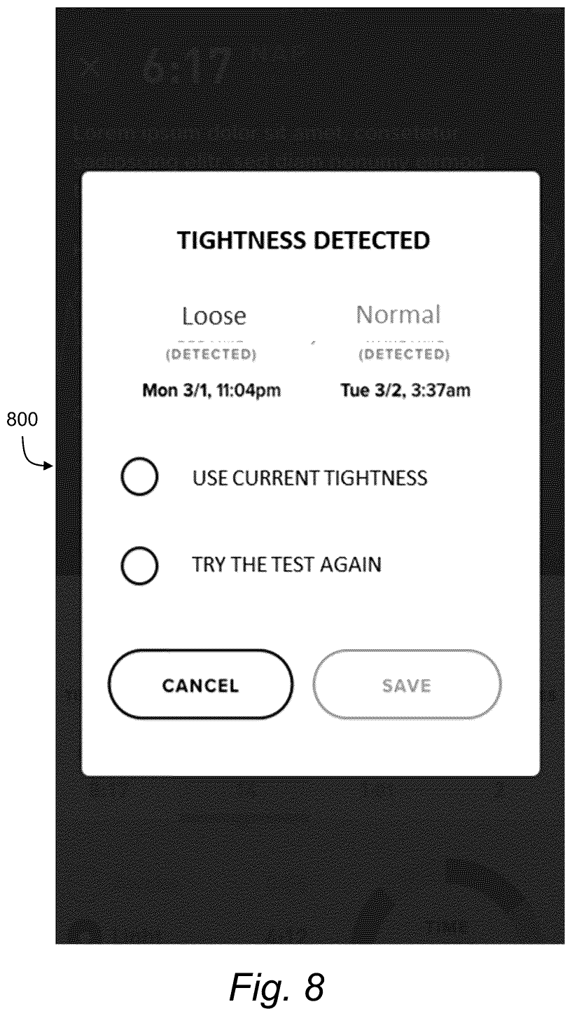

FIG. 8 shows a user interface 800 for user interactions with a device fit protocol, such as any of the user operations described herein. In general, the user interface 800 may be rendered in a smart phone application, web page, or other environment using any of the computing devices described herein. In general, the user interface 800 may be launched, e.g., in response to a user request for a test, in response to an event such as detecting that a user has started wearing a device, in response to a drop of data quality below a threshold, and/or on some predetermined schedule (e.g., once a day or once a week, upon waking, and the like). The user interface 800 may present user options, such as using a current tightness, or running a test again, e.g., after a user has made an adjustment based on device feedback. The user interface 800 may display, e.g., a quantitative and/or qualitative assessment of current fit, and/or a history of fit assessments for the user and device.

In another aspect, the selection of a model or parameters for analyzing fit may depend on where the device is located and/or the type of device (e.g., a device strapped to the body, a device in a garment pocket, an optical sensor, an electrical sensor, etc.). As such, a location detection algorithm may be used to determine a location of the wearable monitor on the body based on data from, e.g., accelerometers, gyroscopes, optical sensors, and other sensors integrated into the wearable monitor, in order to facilitate the selection of a suitable model for evaluating fit. This may be particularly useful where, e.g., the monitor might be deployed on a wrist band or at other body locations where it might be retained, e.g., with an athletic apparel garment such as a sock, shirt, pants, or the like, or some other elastic strap or combination of straps. The location of a monitor such as a photoplethysmography-based heart rate monitor may imply different tension requirements, e.g., where the tension/location combination as a significant impact on the selection of algorithms or models to process data, e.g., to account for different motion cancelation needs at different locations, to support the identification of suitable heart rate calculation algorithms. The location may also more specifically affect the selection and use of different physical models for evaluating fit as described herein.

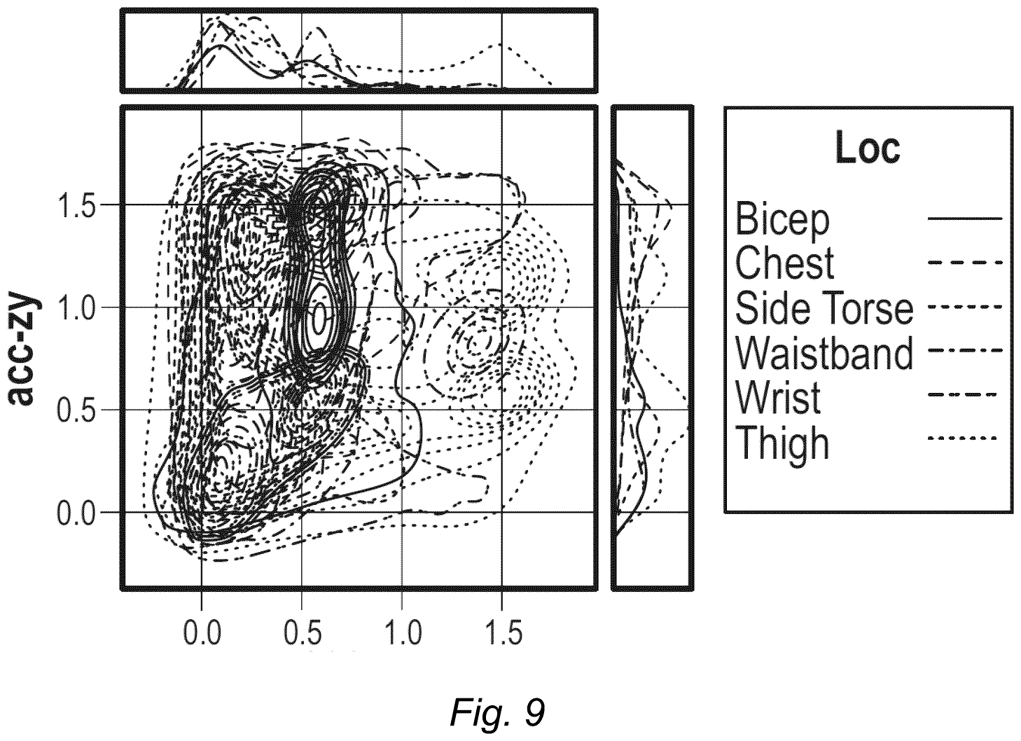

In one aspect, a data driven algorithm may be used to find a location of a wearable monitor without user input by using sensors such as motion sensors and touch sensors within the wearable monitor. In general, the physical orientation and motion of accelerometers and gyroscopes will depend on the location of a monitor on the body. For example, when the user moves forward, a monitor on the torso maintains a fix relation between two set of sensors while when the monitor is on the wrist this relationship changes continuously. FIG. 9 shows accelerometer data patterns for different regions of the body. These empirical patterns of accelerometer data may be used to estimate sensor location, e.g., by mapping motion data from the sensor to one or more regions of these patterns. Similar patterns may be used based on, e.g., magnitude, cross-correlation, rotation (e.g., gyroscopic measurements), and the like to identify sensor location, which may be used, in turn to select suitable data models for evaluating fit as described herein.

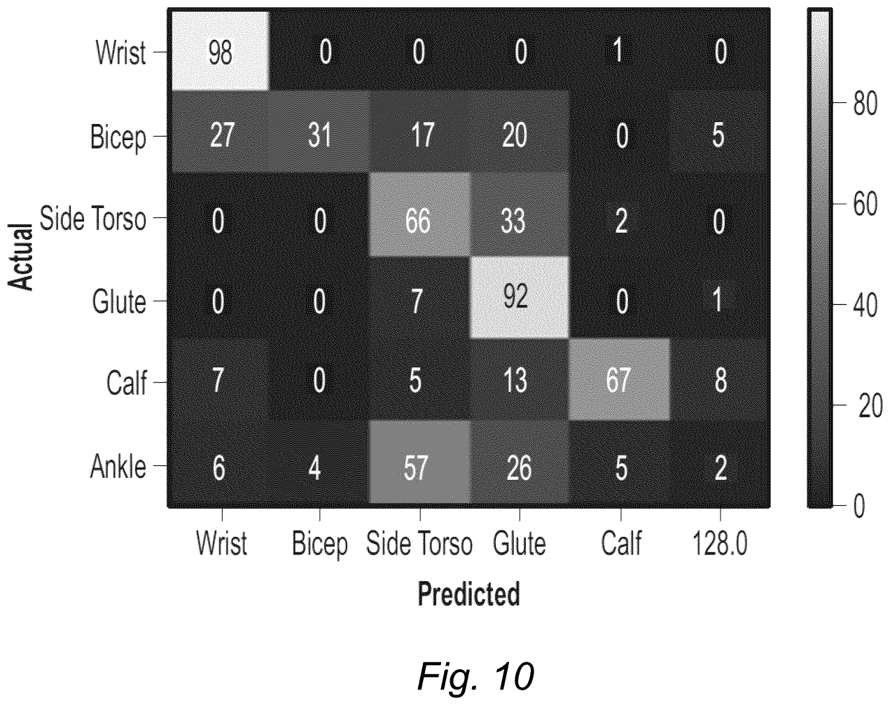

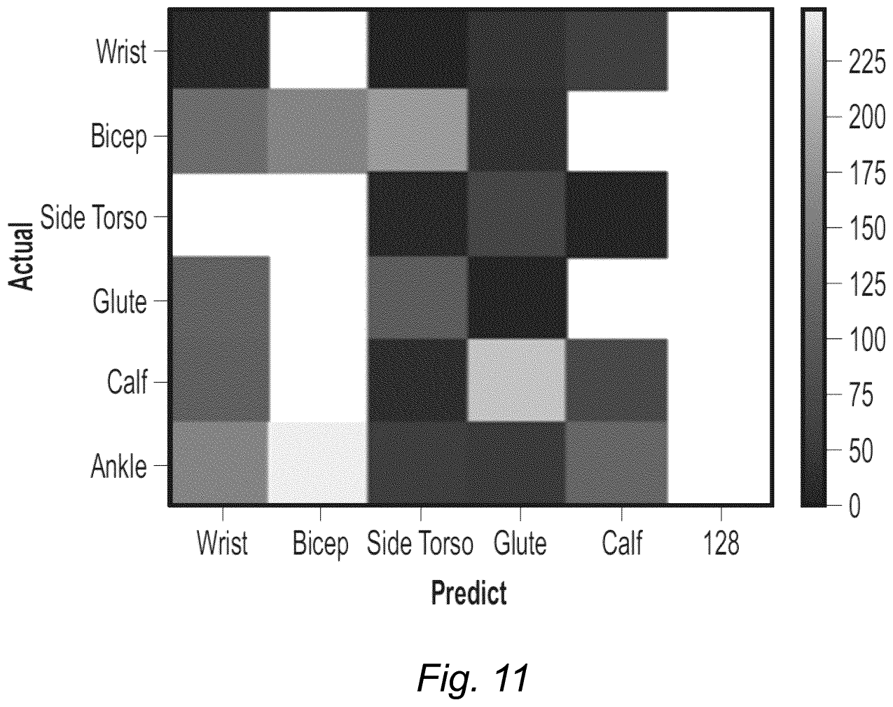

For a range of users and a range of monitor locations, a data driven model may be used to detect location during rest, activities with harmonic motion, and activities with non-harmonic motion. FIG. 10 shows a confusion matrix comparing actual results to predicted results using the data driven model. FIG. 11 shows an amount of time (in seconds of activity) required to achieve a 95% confidence in location for each of the test locations using the techniques described above. In general, these figures illustrate that a data driven model can be derived to usefully detect object location based on sensor data from a wearable monitor over a range of body locations including at least a wrist, bicep, side torso, glute, calf, and ankle. This data may be used to select a resonance model corresponding to the object location for use in estimating strap tension (e.g., method 400 in FIG. 4), or more generally to select processing models, filters, parameters, and the like for processing data from a wearable monitor, particularly in contexts where the monitor is specifically adapted for use on a variety of body locations. For example, it may be determined that the wearable monitor is located on a wrist of the user. A wrist-based model may then be selected for estimating strap tension or otherwise evaluating sensor fit.

More generally, a variety of models are known in the art for determining a location of a device on a user's body, and any such technique may be used, either alone or in combination with the technique described above, to estimate the device location for purposes of choosing suitable models to evaluate fit of a device and/or providing feedback for user adjustments to same.

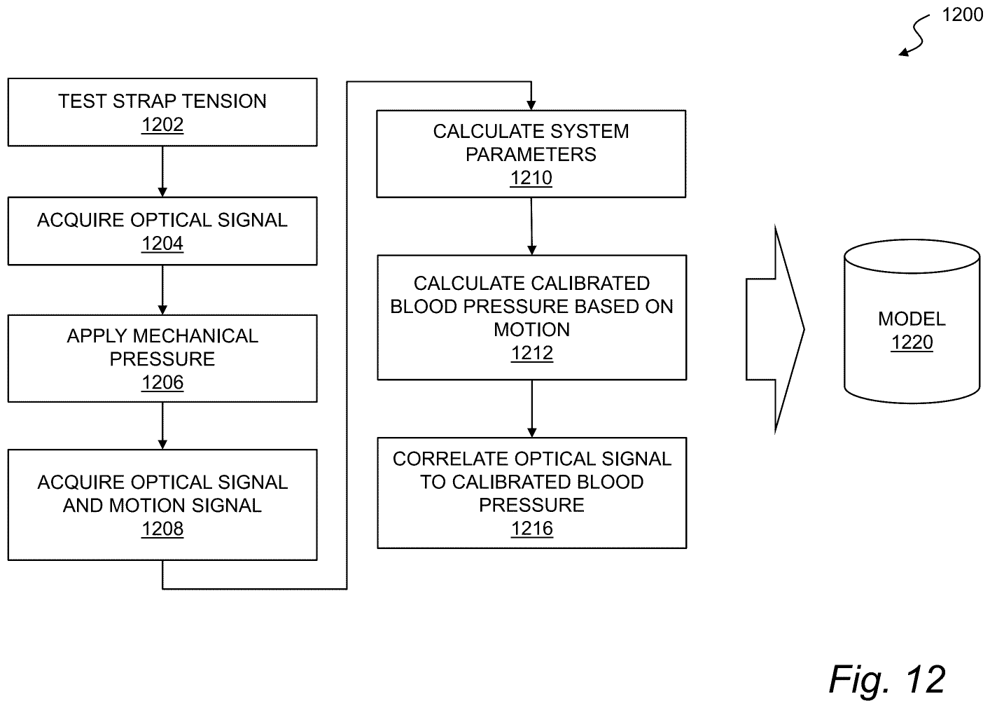

FIG. 12 is a flow chart illustrating a method for creating a model to measure blood pressure. The method 1200 may be realized, e.g., using a wearable device such as a wrist-worn PPG monitor or other wearable fitness monitor or the like. As an initial step, a model for calibrated blood pressure may be generated using a haptic stimulus and motion sensing. In one aspect, physical parameters of a dynamic system for calculating blood pressure can be estimated by varying the pressure applied by the device to a wearer's skin, e.g., using a haptic output, while monitoring motion of the device with accelerometers and/or gyroscopes. For example, where the monitor is secured by a strap or other clastic coupling or the like, parameters for the strap such as tension or a spring constant can be measured (or accurately inferred) using the techniques described herein, e.g., by applying a time varying haptic signal such as a chirp with a haptic actuator of the device, and then measuring responses with a 3-axis motion sensor. With these parameters properly characterized, the haptic signal can be used to measure blood pressure for a measurement volume based on the movement of the wearable device in response to the haptic input. By correlating the calibrated blood pressure measurements from this dynamic model to the optical signals from the PPG sensing system, a model may also be constructed for continuous calculation of calibrated blood pressure based on an optical signal from the PPG sensing system.

It will be understood that the optical signal from a PPG sensing system is generally related to blood pressure. As such, any PPG sensing system can support inferences about blood pressure. However, in the absence of additional modeling (e.g., as described herein), these inferences are uncalibrated. As a significant advantage, the techniques described herein can be used to obtain a calibrated blood pressure measurement in millimeters of mercury (mmHG) that is equivalent to a measurement obtained from a cuff or other device with medical grade accuracy. This calibrated blood pressure measurement can then be used to anchor a measurement of changes in blood pressure that are detected based on an analysis of the optical PPG signal, thus facilitating continuous, calibrated blood pressure measurement with a wearable device.

As shown in step 1202, the method 1200 may begin with testing strap tension for a wearable device. This may include using the techniques described herein to evaluate the mechanical and/or optical coupling between a wearable device and the underlying tissue. For example, testing strap tension may include obtaining an optical signal (e.g., a PPG signal) without vibration, and obtaining an optical signal while vibrating with a haptic device. Where the applied surface pressure (by a haptic actuator) of wearable monitor on the skin begins to occlude blood flow, a photoplethysmography signal will begin to demonstrate a ceiling in response magnitude, appearing, e.g., as clipping or some other flattening phenomenon in the time domain optical signal from one or more photodetectors. As the pressure increases, the peak magnitude at which this clipping occurs in the optical signal will diminish. When the applied pressure fully occludes blood flow through the measurement volume of the tissue, there will be no change whatsoever in the magnitude of the PPG signal. Based on this general phenomenon—changes in the shape of the PPG signal under varying external pressure—the amount of strap tension can be tested by varying haptic loads and observing optical responses, more specifically to ensure that the wearable device is secured with a tension sufficient to cause some clipping, but not sufficient to fully squelch the measured optical signal. Within this range, the system can generally ensure suitable boundary conditions for blood pressure measurement where, e.g., the surface pressure applied by the wearable device is sufficiently high to ensure good mechanical coupling and sufficiently below the blood pressure to ensure a measurable optical response. Within this range, physical parameters can usefully and accurately be extracted for calculation of blood pressure. More generally, testing strap tension may include ensuring that the optical and mechanical coupling of a wearable monitor to a user's skin are optimal as described herein.

A coupling test may be performed automatically before taking a blood pressure measurement, and the user may be guided through a manual tension readjustment process as necessary. Optionally, the wearable device may include any suitable arrangement of electro-mechanical actuators for automatically adjusting to a suitable tension range, or mechanical systems for maintaining tension withing predetermined targets suitable for use with the techniques described herein.

The wearable device may generally include any of the wearable devices described herein, such as a physiological monitor, a photoplethysmography monitor, a heart rate monitor, a wrist-worn monitor with a strap, or some combination of these. The techniques may also or instead be applied in any context where an optical monitoring device such as a PPG system is elastically secured to a user, e.g. to a bicep (with a bicep armband), to a calf, to a waist of a user (with an elastic waistband of athletic apparel), to a torso of user, or at any other suitable physiological monitoring location(s) or context.

As shown in step 1204, the method 1200 may include acquiring optical data such as an optical baseline (also referred to as a baseline signal, baseline PPG signal, or the like) for comparison to the results of the haptic stimulation described below. For example, this may be a time series of optical data acquired from a PPG sensing system. In general, this data may be acquired before or after a mechanical stimulus is applied to the system, but not during the mechanical stimulus, which will disrupt the physiological PPG signal. In either case, the optical data provides a baseline acquired at or near the measured, calibrated blood pressure obtained with the mechanical stimulus. Because a the shape of the PPG signal changes in response to changes in blood pressure, the baseline optical signal can be used for comparison to later-acquired PPG data, and can support a component of the model 1220 that is anchored in the temporally adjacent haptic, calibrated blood pressure measurements, but is adapted for use in identifying changes to the blood pressure relative to the calibrated, haptic measurement.

In general, this optical data acquisition may be performed near the time of the mechanical stimulus of step 1206 so that the calibrated blood pressure determined with the mechanical stimulus can be associated with the baseline optical signal acquired in step 1204, e.g., shortly (or immediately) before or after the mechanical stimulus. In this context, “shortly” means preferably within one minute of, and still more preferably within a few seconds of, the time of the mechanical stimulus. While it is possible to perform all of the following calculations with more temporally distant optical baseline measurements, the relationship of the optical signal to the calibrated blood pressure value will become more tenuous over greater intervals.

As shown in step 1206, the method 1200 may include applying a mechanical pressure to underlying tissue with the wearable device. In general, this may include any technique(s) and hardware/software suitable for generating an applied mechanical pressure or force to the user's tissue. In this context, commercially available haptic actuators can generate a sufficient instantaneous contact force to generate motion artifacts that can be resolved into blood pressure measurements as described herein. For example, a Z30L4B8790008L vibration motor available from Vybronics, Inc. can provide a force of up to one deciNewton at a rotational frequency of about 50 Hz (suitable for sampling at a rate above a Nyquist frequency of 100 Hz), and a rated vibration force of 0.5 Gᵣₘₛ. At a speed of about 25 Hz on a watch-sized, wrist-worn PPG monitor, this actuator was demonstrated to produce measurable motion artifacts suitable for measuring blood flow. Where necessary or helpful, this actuator can be operated at higher rotational speeds, e.g., for greater applied force, greater sampling rates/resolution, and increased decoupling of DC components in detected motion.

It will also be understood that inferences about blood flow and pressure using the techniques herein may depend independently on the speed at which the actuator is operated. For example, for relatively high rotational speeds and a damped response, a DC component may remain in the time-varying contact force experienced by the vasculature that mathematically differs from the mean applied force (e.g., zero, or strap tension) or maximum force. Additionally, hysteresis in the measured tissue volume may change the algorithmic relationship between the mechanics of the vibrating monitor and the occlusion of blood flow, and the nature of this hysteresis may itself vary according to the frequency of the mechanical stimulus. Thus, in general, the nature of the relationship may present challenges for algorithmic modeling, and in some instances, it may be advantageous to perform measurements at two or more different frequencies or otherwise manage and/or vary the haptic drive signals. Thus, in one aspect, the mechanical pressure may be applied as a haptic stimulus using, e.g., a time varying haptic vibration or other mechanical vibration that varies in amplitude, frequency, and/or direction. A haptic chirp output with a time varying frequency, or any of the other haptic signals described herein may be used for this purpose. More generally, haptic vibration in a wearable device can, at appropriate frequency and magnitude, induce measurable motion artifacts correlated to blood pressure, which can support inferences about blood pressure for a user of the wearable device.

As shown in step 1208, the method 1200 may include acquiring an optical signal and a motion signal while applying the pressure to a measurement region as described in step 1206, e.g., with a time varying stimulus provided by a haptic actuator. The motion signal may be acquired from motions sensors such as gyroscopes and accelerometers on a wearable device, or any other suitable motion sensing system(s). For example, this may include data acquisition from a three axis accelerometer and/or a three axis gyroscope, or any other combination of motion measurements useful for creating the model 1220 described herein.

Acquiring the optical signal may include acquiring a signal from a photoplethysmography sensor system of the wearable device during the mechanical stimulus of the wearable device. In general, the mechanical stimulus may be any amount of pressure sufficient to alter blood flow or generate suitable motion artifacts in a detectable manner. As a significant advantage, a properly tensioned wrist-worn strap may be mechanically stimulated with a haptic output device to vary pressure in a manner that produces usefully measurable results that are correlated to blood flow, arterial volume, and/or the like. It will be understood that this second optical signal, acquired during haptic stimulation, may be acquired before or after the first optical signal acquired in step 1204 (or both). Thus, while a particular order is illustrated for convenience in FIG. 12, no such order is required for proper use of the methods described herein. It will also be appreciated that, as described herein, a calibrated blood pressure may be obtained based on the mechanical response to a haptic stimulus, without any optical data. However, the concurrent acquisition of optical data from the photoplethysmography sensing system permits a correlation between optical and mechanical responses that supports inferences about calibrated blood pressure based on the optical signal alone, and can supplement other processing, e.g., by permitting a confirmation that suitable boundary conditions were maintained over a period of haptic stimulus.

As shown in step 1210, the method 1200 may include calculating system parameters. This may, for example, include measuring one or more physical parameters of a system including a wearable device while the device is being worn by a user. The system, for purposes of modeling, may include the wearable device, along with other physical context necessary or helpful for creating a model coupling motion of the wearable device to blood pressure of the user. As described in more detail in the example embodiments of Appendix A, the system may include, e.g., a strap such as a wrist strap, elastic band, or the like that secures the wearable device to a user, along with underlying tissue within or around the measurement volume where (optical) heart rate data and blood pressure measurements are being acquired. For example, useful parameters for a physics model may include a strap tension, a strap spring constant, and a tissue elasticity (e.g., of the skin, or of tissue adjacent to the wearable device). It will be understood that other parameters may also or instead be used in this physical model, such as elasticity of vasculature, strap elasticity, surface pressure, instantaneous contact force, and so forth. In another aspect, any one or more of these parameters may be modeled as a lumped parameter, such as a lumped elasticity or spring constant for the entire system, where they behave in a suitable linear manner.

In one aspect, calculating the system parameters may include automatically measuring one or more of the parameters using, e.g., any of the techniques described herein to estimate, calculate, or otherwise resolve physical parameters or other parameters for the system. For example, the dynamic system may include the wearable device and the measurement volume (of the user), and a physical parameter may include a tissue constant for tissue in the measurement volume of the dynamic system. However, other parameters may also or instead be measured using haptic stimulus and response, such as a tension of a strap securing the wearable device to the user or a spring constant for a system including the wearable device and a strap that secures the wearable device to a user.

In one aspect, system parameters may be calculated using inputs and outputs of the wearable device. For example, an output may include a stimulus such as a mechanical stimulus from a haptic output device associated with the wearable device, and an input may include motion data form one or more motion sensors. In this case, measuring one or more physical parameters may include calculating at least one of the one or more parameters based on a motion response of the wearable device to a haptic stimulus. This process may be accompanied by, or guided with, user instructions provided through any suitable interface. For example, if the measurement is most accurate when a user is at rest, then the user may be instructed to remain at rest while this measurement is being taken. The haptic stimulus may be a time varying haptic vibration such as a mechanical vibration that varies in amplitude, frequency, and/or direction. A haptic chirp output, or any of the other haptic signals described herein may be used for this purpose. The motion response may be measured, in turn, using any suitable sensors associated with the wearable monitor, such as an accelerometer, a multi-axis accelerometer (e.g., a three-axis accelerometer), a gyroscope, a multi-axis gyroscope, or any combination of these or other motion sensors. This technique may also or instead be used to measure the physical parameters of the skin. For example, in one aspect, measuring one or more physical parameters may include skin elasticity. This technique may also or instead be used to measure the physical parameters of the skin. For example, in one aspect, measuring one or more physical parameters may include measuring a skin elasticity. In another example, elasticity or spring constants for a strap and underlying tissue can be measured based on motion responses to a haptic stimulus.

Some parameters may require or benefit from additional data. For example, in order to measure tension in a strap, the method 1200 may include measuring optical coupling and mechanical coupling between the wearable device and the underlying measurement tissue. This measurement may employ the baseline optical signal (without haptic stimulus) described above, along with a second optical signal during haptic stimulus. By comparing these two signals, the degree of optical and mechanical coupling can be determined, and used to resolve a tension in the strap. A number of other techniques for calculating tension in a strap are described herein, and may be used to calculate a tension parameter for a physical model of a wearable device. More generally, any technique for resolving optical signals during vibration, optical signals without vibration, motion signals during vibration (including accelerometer data and gyroscope data), and the like, may be used to calculate strap tension and other system parameters for use in a physical model as described herein.

Other techniques may also or instead be used to measure physiological parameters. For example, in one aspect, measuring one or more physical parameters may include evaluating at least one of the physical parameters with a machine learning model based on inputs from one or more sensors of the wearable device. That is, a machine learning model may be trained to associate combinations of mechanical stimuli and measured responses for a particular system (such as a wrist-worn physiological monitoring device) with particular parameters or parameter ranges. This trained model may be used, in turn, to estimate one or more system parameters based on a particular set of haptic signals and motion responses. It will be understood that this may include multiple models, e.g., to account for different styles of monitors, straps, and the like, which may be specified as a user input or detected from the device. In another aspect, one or more features may be inferred by the machine learning model, in which case a separate measurement or input of these additional system variables may be omitted.

In another aspect, one or more parameters may be measured directly. While commercially available haptic devices provide a small, convenient, and inexpensive actuator for securing the necessary data to properly model a dynamic system for measuring blood pressure with a wearable device, a variety of other sensors and sensing techniques are also known in the art, and may be adapted to obtain suitable model parameters for use with the techniques described herein. For example, tension or surface pressure may be measured using a force sensor, pressure sensor, or the like on the wearable device, and used to calculate system parameters for a physical model for evaluating blood pressure. In another aspect, strap tension may be measured using a force sensor, strain gauge, or the like coupled within the strap to directly sense strap tension. Other parameters such as device weight, strap tension, and the like may also or instead be manually measured using any suitable gauges, and then provided as manual inputs by a user.

As shown in step 1212, the method 1200 may include calculating a blood pressure for the measured tissue volume. As described above, a haptic stimulus can be used to generate artifacts in a wearable device correlated to blood pressure. For example, as described in U.S. Prov. App. No. 63/596,469, a time-varying blood pressure may be calculated based on motion artifacts during haptic stimulus as follows:

$$β ( t ) = {m}_{strap} A ( t ) + {m}_{strap} g + {T}_{o} - {k}_{total} x ( t ) - {r}_{h} {m}_{h} \frac{d [ cos ( \hat{H} ( A ) ( t ) ) ]}{dt} \qquad [ Eq . 1 ]$$

where:
mₛₜᵣₐₚ is the mass of the wearable device,
A(t) is time-based data from an accelerometer in the device,
g is the acceleration due to gravity,
kₜₒₜₐₗ is a lumped elasticity parameter for the strap and tissue,
Tₒ is the tension in the strap (at rest),
x(t) is the z position of the device (calculated based on accel/gyro data),
rₕ is the effective radius of the center of mass of the haptic device,
mₕ is the mass of the haptic device, and
H( ) is the phase of the Hilbert transform.

It will be noted that in this expression, the device is assumed to be flat on top of the wrist, which is also the nature position for conventional cuff blood pressure measurements. However, mathematical accommodations may also be made for other orientations, e.g., to account for the direction of acceleration due to gravity. It will also be noted that all of the constants in this expression are either known or can be calculated using the techniques described herein. For example, the mass of the device, the mass of the haptic device, and the effective radius of the center of mass for the haptic device can be measured (or obtained from a manufacturer) and provided as constants to this equation. The motion data, A(t) and x(t), can be obtained from motion sensors on the device as a time series of data. The Hilbert transform is a known function for transforming a time series of data. And acceleration due to gravity is a known constant. This leaves tension and lumped elasticity, which can be derived from other available information.

As described in greater detail in U.S. Prov. App. No. 63/596,469, the lumped elasticity, kₜₒₜₐₗ, may be calculated based on motion data as follows:

$${k}_{total} = \qquad [ Eq . 2 ]$$

$${E}_{t} [ \frac{1}{hp ( t ) * 𝒢 ( t )} {m}_{strap} ( hp ( t ) * \frac{dA ( t )}{dt} ) - {r}_{h} {m}_{h} ( hp ( t ) * \frac{{d}^{2} [ cos ( \hat{H} ( A ) ( t ) ) ]}{{dt}^{2}} ]$$

where:
hp(t) is a high pass filter (with cut-off frequency>1 Hz),
* is a convolution (and therefore β(t)*hp(t)=0), and
Eₜ[ ] is the expectation operator (expected value for a random variable).

As also described in U.S. Prov. App. No. 63/596,469, the tension for the strap that secures the monitor to the body can be calculated based on motion data. In this case, a number of assumptions may be used to support a derivation of the relationship between motion data and tension. In particular, by assuming that the elastic band securing the device is within a linear elastic region, and by further assuming that the elastic band is tight enough so that the monitor/strap does not slide on the skin during vibration, the following relationship may be derived between motion data and strap tension:

$${T}_{o} = Δ L \cdot w \cdot {E}_{t} [ \frac{{m}_{strap} \frac{{dA}_{y} ( t )}{dt}}{cos ( θ ) {G}_{x} ( t )} ] \qquad [ Eq . 3 ]$$

where:
ΔL is the change in the length of the elastic band from rest,
w is the circumference of the band about the wrist (or other body part),
θ is the angle between the wearable device and the skin,
A_{y}(t) is the time-based accelerometer signal in the y direction, and
Gₓ(t) is the time-based gyroscope signal in the x direction.

This leaves an expression (Eq. 1, above) that relates calibrated blood pressure, β(t), to a time series of measurements by an accelerometer and a gyroscope in the wearable device. In general, the measurements that appear most correlated to accurate estimation of blood pressure (e.g., yielding the most accurate model results) appear to be the z-axis gyroscope measurement, and the z-axis and y-axis accelerometer measurements (using the coordinate system illustrated in FIG. 6). However, other motion data may be available from sensors in a wearable device, and may be used to refine or modify a physical model for measuring blood pressure as described herein.

It will also be noted that, once the model 1220 has been calibrated by evaluating any need parameters, the blood pressure may be extracted from the same motion signal, A(t), that was used to perform the calibration. Thus, a calibrated blood pressure measurement may be obtained concurrently with parameterizing the model, and/or calculating other system parameters as described in step 1210, above. In another aspect, additional mechanical stimulus may be applied while additional motion data is acquired, and blood pressure may be calculated by applying the new motion data to the model 1220. While the latter approach can significantly simplify subsequent blood pressure calculations, it may also be useful to repeat the calibration by recalculating the underlying physical parameters, e.g., to ensure that the modeled system has not changed due to changes in the position of the strap, tension on the strap, or other contextual or environmental changes that might affect the parameters of the model 1220.

By properly evaluating and scaling the constants in this equation, calibrated blood pressure measurements may be calculated that correspond to conventional pressure cuff measurements. It will be understood that the result of this equation is a time series of blood pressure measurements. In order to report a more conventional assessment of blood pressure, the measurement may be converted to a conventional cuff measurement, e.g., by analyzing the resulting waveform to identify maxima (systolic blood pressure) and minima (diastolic blood pressure). These two numbers (diastolic and systolic blood pressure, typically reported in mmHG) may be provided as an instantaneous measurement, or may be averaged over a number of heartbeats, or a number of seconds or more to obtain a more consistent, repeatable result.

Using the foregoing techniques, a model 1220 for calculating blood pressure may be obtained. This includes a first aspect of the model 1220 for calculating calibrated blood pressure based on mechanical stimulus, e.g., using Eq. 1 above. The model 1220 may be recalibrated under haptic (or other mechanical) stimulus from time to time, e.g., to account for strap removal, physical location shifting, changes in skin condition, and so forth. This recalibration may be performed automatically, e.g., on a predetermined schedule or in response to conditions suggesting that the strap has shifted or that blood pressure measurements are meeting or exceeding thresholds for expected ranges. In another aspect, a recalibration may be performed in response to a user input or command to perform a recalibration, e.g., prior to capturing a systolic/diastolic blood pressure measurement for display or periodic reporting.

While the foregoing techniques permit measurement of a calibrated blood pressure under haptic stimulus, this technique may consume substantial power to operate the haptic actuator and calculate corresponding parameters. Haptic stimulus may also create significant disturbances to a user if deployed for continuous blood pressure monitoring. Thus a second aspect of the model 1220 may support calculating a continuous and/or time varying blood pressure based on optical signals from a PPG sensing system or the like, as described in greater detail below.

As shown in step 1216, the method 1200 may include correlating the data in the optical signal to the calibrated blood pressure calculated from the mechanical stimulus. In one aspect, the initial calibrated blood pressure calculated during haptic stimulation may be mapped to or associated with the optical baseline acquired shortly before or after the haptic stimulation. If the blood pressure remains the same, then the pulse shape of the PPG signal will also remain the same, absent changes it strap tension, strap position, and so forth. For continuous monitoring of a time-varying blood pressure based on the optical signal, however, additional processing may be required. In order to correlate the optical PPG signal to a blood pressure that varies over time, the shape of the PPG pulse may be characterized, and changes to the PPG pulse shape may be associated with changes in the blood pressure.

This may include an initial step of characterizing the pulse shape of the baseline optical signal that is associated with the calibrated blood pressure measurement. A number of characteristic features of a PPG signal are known in the art, and may be used to characterize the pulse shape for the purposes contemplated herein, e.g., to provide parameters for detecting blood pressure variations. In one aspect, one or more individual metrics may be used to estimate changes in blood pressure. For example, changes in amplitude of the PPG signal or pulse length may be used to directly estimate changes in blood pressure relative to the baseline optical signal.

In another aspect, a more complex dynamic model may be created, e.g., using an aggregation of pulse characteristics. For example, one useful characteristic is the height of the pulse dicrotic notch. The dicrotic notch is a small dip or downward inflection that occurs just after the peak of the pulse wave, which represents the closure of the aortic valve and precedes the dicrotic wave (a smaller secondary upward deflection). This may be measured as a zero crossing in the volume pulse graph between systolic and diastolic peaks. Other features may also or instead be used, such as the slope before the systolic peak, the slope after the diastolic peak, a height of the systolic peak, a height of the diastolic peak, an interval between the systolic peak and the notch, an area under the pulse, an area under the pulse between the systolic peak and the notch, a pulse width, a pulse area, a peak-to-peak interval, and the like. Other derived metrics such as an augmentation index and an arterial stiffness index may also or instead be used. For example, the augmentation index measures pressure wave reflections in the arteries, and is used as an indication of arterial stiffness. Similarly, the Large Artery Stiffness Index is a measure of the stiffness of large arteries, and can be inferred from aspects of the PPG pulse. More generally, any feature or characteristic of the PPG pulse that can be objectively calculated, and that is correlated to changes in blood pressure, may be used as a parameter for detecting blood pressure variations.

A number of these parameters may be calculated for a first pulse sample from the optical baseline (where blood pressure is the calibrated blood pressure) and a second pulse sample from a subsequent point in time. A vector may be created for each pulse sample, and then weighted on a feature-by-feature basis according to a priority of importance for each feature in estimating changes in blood pressure. The weighting may depend on the value of other parameters, and in one aspect may be selected using a machine learning model trained to rank or otherwise prioritize the use of the available parameters based on the characteristics of the baseline optical signal and/or current optical signal. The similarity of the second pulse to the first pulse can then be calculated as the distance in the vector space between the weighted feature vectors. The similarity between the pulses (based on the vector distance) may be used to create a dynamic model (also referred to as a second aspect of the model 1220) for determining whether (and by how much) the current blood pressure is greater or less than the calibrated blood pressure associated with the baseline optical signal. In one aspect, the dynamic model may be adapted around known relationships between changes in pulse shape and changes in blood pressure. The dynamic model may also or instead be calibrated or refined for a user based on additional cuff measurements, haptic calibrated measurements, and/or otherwise trained using data from individuals with known changes to blood pressure, so that the relationship between the feature vectors for the baseline pulse shape and the subsequent pulse shape can be used to infer changes in blood pressure with greater accuracy.

More generally, any machine learning or statistical modeling techniques that can associate changes in pulse shape with changes in blood pressure (e.g., from a calibrated baseline measurement) may be used to support a dynamic model that predicts blood pressure changes based on the extracted PPG features, and the model 1220 is intended to include all such models that can accurately predict such changes. By way of non-limiting example, Chowdhury, et al., “Estimating Blood Pressure from the Photoplethysmogram Signal and Demographic Features Using Machine Learning Techniques,” Sensors, 2020 Jun. 1; 20(11):3127 (doi: 10.3390/s20113127. PMID: 32492902; PMCID: PMC7309072), describes machine learning techniques for selecting relevant features and continuously monitoring changes in blood pressure. This document is hereby incorporated by reference in its entirety.

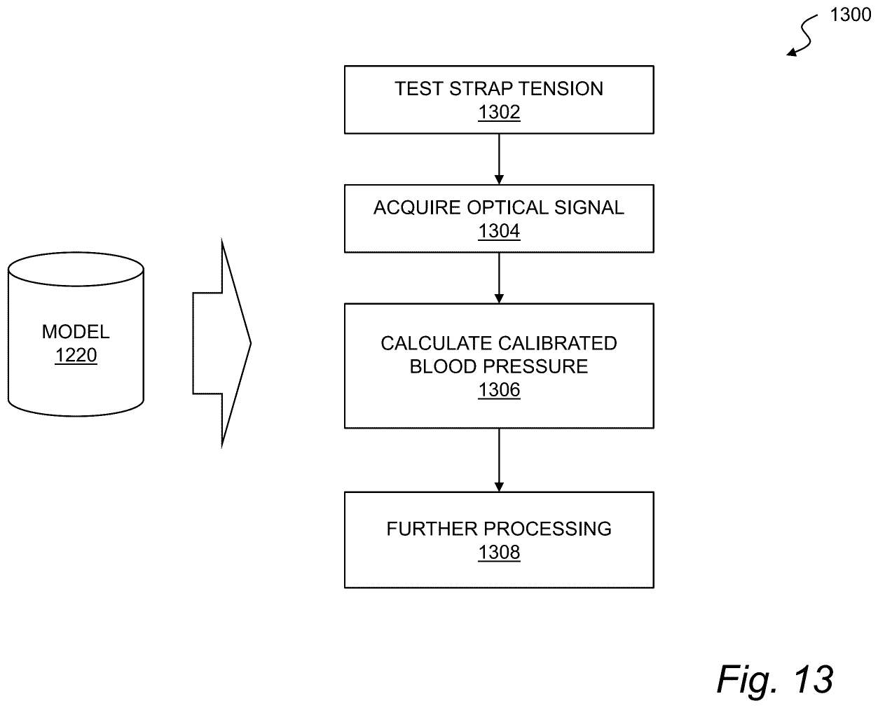

FIG. 13 is a flow chart illustrating a method for using a model to calculate calibrated blood pressure from an optical signal. The method 1300 may use a blood pressure model 1220 such as any of the models described herein.

As shown in step 1302, the method 1300 may include testing strap tension, for example using any of the techniques described herein. This may include generally evaluating the quality of optical and/or mechanical coupling using the techniques described herein. This may also or instead include evaluating whether the current strap tension and/or location appears similar to the conditions under which the model 1220 was calibrated. While it may not be necessary to test strap tension before each blood pressure measurement, this can help to ensure that the measurement conditions are suitably similar to the calibration conditions present when the model 1220 was created.

As shown in step 1304, the method 1300 may include acquiring an optical signal, such as a time series of optical intensity measurements from a PPG sensing system or the like.

As shown in step 1306, the method 1300 may include calculating a calibrated blood pressure based on the optical signal. In general, this may include applying the time series of optical signal data from the PPG sensing system to the model 1220, for example by extracting features from the optical signal data and calculating a similarity or distance to the shape of the baseline signal. This can be used to estimate a change in blood pressure from the baseline blood pressure (which is equal to the calibrated blood pressure obtained during haptic stimulus) to the current blood pressure, which can be added to or subtracted from the calibrated blood pressure to obtain the current blood pressure. In one aspect, calculating the calibrated blood pressure may include continuously calculating a blood pressure based on the calibrated blood pressure and the optical data, and then identifying systolic and diastolic peaks in the continuous blood pressure. In another aspect, the method 1300 the model 1220 may provide independent adjustments to the systolic and diastolic values obtained with the calibrated blood pressure measurement. In either case, the current blood pressure may be reported to a user as an instantaneous measurement, or as an average for a number of measurements over a predetermined time period, a moving average over a predetermined interval, and so forth. In another aspect, the model 1220 may include a machine learning model trained on the foregoing data to output blood pressure changes based on feature vectors obtained from the (baseline and/or current) optical PPG signals.

As shown in step 1308, the method 1300 may include a variety of practical refinements to and uses of a calibrated blood pressure. For example, this may include storing the measured blood pressure, either as a single measurement, or with a collection of historical measurements of the user's blood pressure for a longitudinal study. In order to facilitate accuracy and consistency, measurements may be taken under specific, repeatable testing conditions, e.g., while sitting after at least five minutes of inactivity, and with the palm of the hand resting in a predetermined location (e.g., on the lap, on an armrest, etc.) and in a predetermined orientation (e.g., facing up, facing down, etc.). The user may be automatically prompted to take a measurement by presenting instructions to the user on a device such as a smart phone associated with the wearable device. This may occur on a specific schedule, e.g., at a particular time each day (or on one or more days during the week). In another aspect, a user prompt may be generated in response to detecting that the user is at rest in a manner suitable for consistent blood pressure measurement, and the prompt may be presented to the user on a smart phone, tablet, or other personal computing device, or by haptically signaling to the user with the wearable monitor.

In another aspect, the method 1300 may include some combination of these techniques. For example, within a window extending for one hour from waking up, a heart rate and activity of the user may be monitored to identify a suitably long period of inactivity such as ten minutes in a chair or lying reclined. Upon detecting such a period within the post-wakeup window, the user may be prompted to initiate a blood pressure measurement, and optionally provided with instructions that guide the user through an accurate measurement protocol. In another aspect, the blood pressure measurement may be automatically performed when conditions are suitable, or automatically performed on some predetermined schedule and then analyzed to ensure that the measurement was taken under conditions suitable for accurately reporting a calibrated blood pressure. In another aspect, blood pressure measurements may be taken on demand, and the user may be similarly guided to achieve consistent and accurate results.

Where blood pressure measurements are spaced apart by some substantial time interval, or where conditions have changes (e.g., the strap has been removed and replaced), a haptic recalibration may automatically be performed, or the user may be prompted to manually initiate a recalibration.

In another aspect, the blood pressure may be displayed, e.g., on a computer device associated with the user, on the wearable device, or on a website interface or the like accessible to the user, and/or stored on a server for subsequent use. In another aspect, the blood pressure may, either alone or in combination with other health data (including historical blood pressure measurements) be used to generate alerts, recommendations, or the like for the user to promote awareness and wellness, or to initiate medical care where appropriate. The blood pressure may also or instead be used with other data to evaluate general wellness, and/or to provide a daily health report along with other health metrics such as respiration rate, body temperature, resting heart rate, heart rate variability, sleep duration, and so forth, as well as derived wellness statistics such as a stress score, a strain score, a sleep quality score, a recovery score, and the like for a current or historical time period. More generally, any medical, fitness, health and wellness, or other uses of an accurate, calibrated blood pressure measurement may be facilitated by a wearable physiological monitoring device that is configured as described herein to obtain calibrated blood pressure measurements for a user that accurately correspond to conventional cuff pressure measurements taken by medical professionals.

According to the foregoing, in one aspect, the methods and systems described herein may include creating a blood pressure model 1220 (such as any of the dynamic models or the like described herein), storing the blood pressure model on a computing device, and using the blood pressure model to calculate blood pressure for a wearer. This may include calculating blood pressure based on measured motion, and/or calculating blood pressure based on an optical signal, e.g., once the optical PPG signal has been correlated to a calibrated blood pressure obtained with haptic stimulus. Thus in one aspect, there is disclosed herein a method comprising: calibrating a model for a wearable device while the wearable device is placed for use on a user, the wearable device including a haptic actuator and a motion sensor, and the model expressing a calibrated blood pressure for a wearer of the wearable device over time as a function of a number of constants and a motion signal for the wearable device over time; operating the haptic actuator with the driving function; measuring the motion signal for the wearable device; calculating the calibrated blood pressure with the model over a period of time; and analyzing the calibrated blood pressure over the period of time to identify a systolic blood pressure and a diastolic blood pressure for the user.

In general, the model may be stored on and/or executed on the wearable device, a personal computing device coupled to the wearable device, a server or other remote computing resource, or some combination of these. The model may, for example, include a physical model that characterizes physical behavior of a system including the wearable device, an elastic band securing the wearable device to a user, tissue of the user, and so forth. The model may also or instead include a dynamic model that characterizes responses to time-varying variables such as a haptic stimulus, measured motions, or optical data from a PPG sensing system.

In one aspect, there is disclosed herein a system comprising: a wearable device including a strap for coupling the wearable device to a user, a haptic output device, and a plurality of motion sensors; a memory storing a model for the wearable device, the model calibrated with the wearable device secured to the user with the strap, and the model expressing a calibrated blood pressure for a wearer of the wearable device over time as a function of at least a number of constants and a motion signal for the wearable device over time; and a processor configured by computer executable code stored in a non-transitory computer readable medium to perform the steps of: operating the haptic actuator with the driving function, measuring the motion signal for the wearable device, calculating the calibrated blood pressure with the model over a period of time, and analyzing the calibrated blood pressure over the period of time to identify a systolic blood pressure and a diastolic blood pressure for the user.

In another aspect, a method disclosed herein includes measuring a calibrated blood pressure in mmHG; acquiring a first PPG signal with a wearable monitor, the first PPG signal temporally proximal to measuring the calibrated blood pressure (e.g., shortly before or after measuring the calibrated blood pressure), the first PPG signal including a first pulse; associating a first shape of the first pulse with the calibrated blood pressure; acquiring a second PPG signal with the wearable monitor, the second PPG signal including a second pulse temporally distal to measuring the calibrated blood pressure; detecting a change in pulse shape from the first shape of the first pulse to a second shape of the second pulse; estimating a change in blood pressure from the calibrated blood pressure based on the change in pulse shape; and calculating an estimated blood pressure for a user of the wearable monitor based on the calibrated blood pressure and the estimated change in blood pressure. The calibrated blood pressure may include a systolic blood pressure and a diastolic blood pressure. The change in blood pressure may include a first change in a systolic blood pressure and a second (independent) change in diastolic blood pressure. The calibrated blood pressure may be calculated using a haptic actuator of the wearable monitor as described herein.

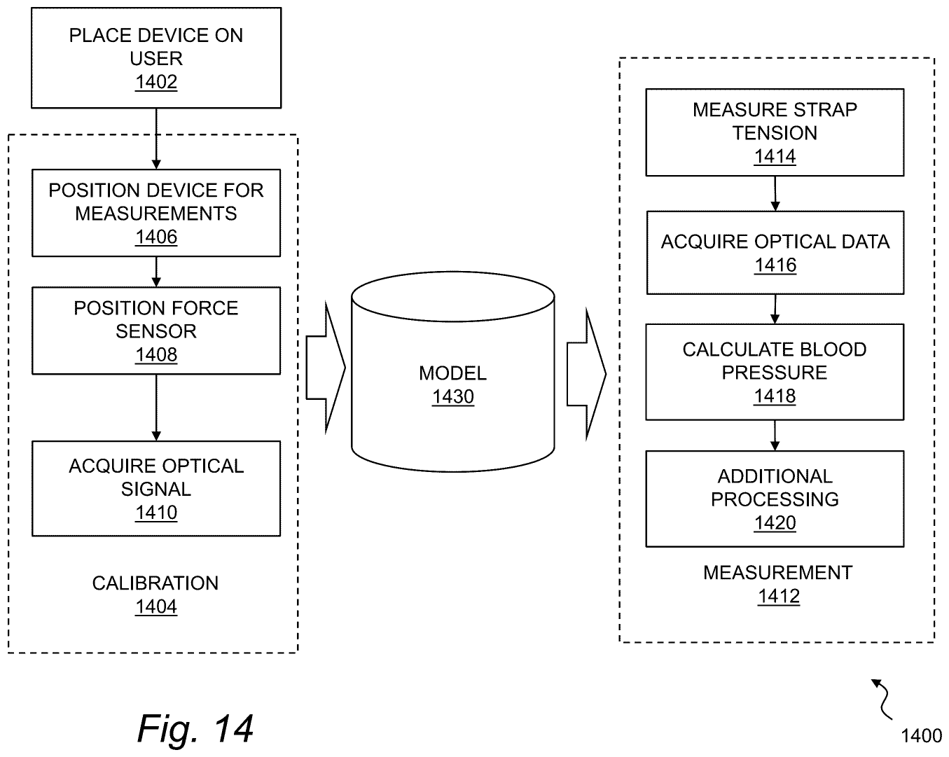

FIG. 14 is a flow chart illustrating a method for measuring blood pressure. In general, by positioning an optical sensing system (such as an optical sensing system of a wearable physiological monitor) over a user's radial artery, and then acquiring calibration data over a range of known, applied pressures, a model can be derived for continuous, calibrated blood pressure monitoring. By adding a force sensor to a photoplethysmography device or other optically based, wearable physiological monitoring system, suitable calibration data can be acquired and applied to adapt the wearable device for use as a blood pressure monitor for real time, calibrated measurements of diastolic and systolic blood pressure.

As shown in step 1402, the method 1400 may include placing a wearable device on a user. For example, this may include securing a physiological monitor or other wearable device to the user, e.g., with an elastic strap, an adjustable strap, or other suitable mechanism(s) for securing monitoring hardware about a wrist or other location on the user's body. The wearable device may, for example, include a wearable continuous physiological monitor or wearable photoplethysmography device such as any of the wearable devices described herein. In one aspect, the optical sensor may be a multi-sensor system and may include, for example, at least an illumination source, a first photodetector positioned to detect light from the illumination source directed toward a radial artery of the user when the wearable device is placed for use, and a second photodetector positioned to detect light from the illumination source directed away from the radial artery of the user when the wearable device is placed for use. In this configuration, the device may usefully isolate optical artifacts resulting from blood traveling through the radial artery—the target for measuring blood pressure—from other optical artifacts that result from changes in blood volume and blood flow in vasculature of the surrounding tissue.

As shown in step 1404, the method 1400 may include calibrating the wearable device for blood pressure measurements. This may, for example, include a number of steps to acquire data to configure, parameterize, or otherwise derive a model for continuous blood pressure monitoring as contemplated herein. These steps may be performed, e.g., on a wearable device, on or in cooperation with a computing device associated with the wearable device (such as a smart phone, tablet, laptop, or the like), and/or on or in cooperation with a server coupled in a communicating relationship with the wearable device. For example, this may include steps 1406-1410 below, or any other suitable combination of steps and measurements useful for calibrating a monitoring device to provide continuous or intermittent non-invasive blood pressure measurements that are calculated based on optical signals, and that correspond to a conventional measurement of blood pressure in mmHG from a cuff or other medical grade measurement device.

As shown in step 1406, the calibration 1404 may include positioning the device for measurements. This may include placing an optical sensor of the wearable device in a position over a radial artery of the user while the wearable device is secured (e.g., about the user's wrist) with the strap. For example, the wearable device may usefully be positioned with a first optical path that passes from an optical source through the radial artery to a first sensor, and a second path from the optical source to a second sensor of the optical sensor that does not pass through the radial artery. In this manner, optical data related to the radial artery can be isolated (e.g., by subtracting the signal from the second optical sensor) from other optical data related to cardiac activity in the surrounding vascular network. It will be understood that other arrangements may also or instead be used to locate a radial artery, e.g., by wavelength or time multiplexing two optical sources in different paths to a single optical detector.

In order to position the optical sensor, a user interface may be configured for a user to review data from the optical sensor(s), and/or to provide instructions in the user interface that guide the user to properly position the wearable device (or more specifically, the optical sensor of the wearable device) to a location suitable for blood pressure measurements. For example, optical measurements, e.g., for two different sensors on two sides of a light source, will generally be similar and/or symmetrical when positioned over tissue with underlying vasculature. However, an infrared signal can penetrate deeper into tissue than other frequencies (e.g., green or red), and when passing through a radial artery, will change in intensity as the diameter of the artery changes. Thus, an infrared signal passing through the radial artery will provide a significantly different return signal than other infrared signal paths, and a pair of infrared measurements at two detectors will become asymmetric (in a manner that green or red signals will not) when one path passes through a radial artery. This difference may be used to provide instructions to reposition a wearable device, e.g., by rotating the device and strap around a wearer's wrist, until the expected or desired signal disparity is present. This may include binary instructions (e.g., “rotate” or “stop”) or relative instructions (e.g., “You are close to the correct position. Rotate more slowly.”). As noted above, this difference also advantageously supports improved signal resolution by permitting subtraction of an infrared signal that does not pass through the radial artery in order to remove artifacts associated with vasculature and other time-varying tissue properties.

According to the foregoing, placing the optical sensor for blood pressure measurements, as described herein, may include guiding the user to position the wearable device about a wrist of the user by presenting instructions to the user in a user interface, e.g., instructions to continue moving/rotating the sensor position until the target optical signals are received. By way of non-limiting example, the wearable device may be guided to the position over the radial artery by providing illumination with an optical source, receiving a first optical signal at a first optical sensor, receiving a second optical signal at a second optical sensor, and guiding a user to move the optical sensor toward the position over the radial artery based on a difference between the first optical signal and the second optical signal.

As shown in step 1408, the calibration 1404 may include positioning a force sensor to measure a contact force between the wearable device and the user. In embodiments where the wearable device has a built in force sensor for detecting contact force with the user's tissue, this positioning step may be performed concurrently with placing the wearable device on the user, or rotating the wearable device into a position over the radial artery. However, a system may also or instead include a removable and replaceable force sensor for use in calibrating an optical device for blood pressure measurements. Thus in one aspect, the force sensor may be removably and replaceably coupled to the wearable device, e.g., by snapping or sliding the force sensor onto a housing of the device, or by inserting the force sensor between a portion of the housing and the tissue (preferably in a location that does not obstruct or interrupt optical coupling between the optical sensor and the user's tissue).

For example, the force sensor may be a disposable pressure sensor removably affixed to an exposed surface of the wearable device and accessible to a user when the wearable device is placed for use on the user. In another aspect, the force sensor may be incorporated into a removable recharging battery such as the battery 106 depicted in FIG. 1, or into the wearable device itself, such as any of the sensors 314 described with reference to FIG. 3. Thus the battery 106 may be, or may include a force sensor that is removable from and replaceable to the device 104.

In one aspect, the force sensor may include electronics for measuring a force between the wearable device and user tissue, as well as accompanying electronics for recording force measurements and/or transmitting force measurements as appropriate to another device for use in calibration as described herein. In another aspect, the force sensor may be configured to measure force between a finger of the user and the wearable device when the user pushes the wearable device toward the user's tissue. This latter configuration may be used, e.g., with a tension measurement by the wearable device to support a calculation of a net contact force resulting from a combination of strap tension and applied force. It will be appreciated that the terms force and pressure are sometimes used interchangeably herein, although they are different quantities of measure with different units, and it may be necessary to convert between force and pressure when performing calculations or otherwise using force/pressure data for the purposes described herein. The techniques for converting between force and pressure are well known in the art, and need not be repeated here.

As shown in step 1410, the calibration 1404 may include obtaining calibration data for a blood pressure measurement of the user with the wearable device. For example, this may include acquiring optical data from the optical sensor and force measurements from the force sensor while applying a range of forces to the skin of the user with the wearable device. The optical data may, for example, include an optical signal from a photoplethysmography heart rate monitor, or another optical sensor system such as a separate infrared sensing system for illuminating user skin and measuring reflected light.

In one aspect, the calibration data may include an oscillometric envelope derived from the optical data. The term “oscillometric envelope” generally refers to an envelope for an oscillating pattern of cardiac activity as pressure is gradually increased or decreased over a range of arterial pressures. While this is conventionally measured with pressure changes applied through a blood pressure cuff, measurements for an oscillometric envelope can also be obtained by applying surface pressure over the radial artery while acquiring optical data from an infrared transmitter and receiver with an optical path through the radial artery. In this context, the calibration data may include a waveform (or other min/max value or the like) for the optical signal captured over a range of pressures including at least a first pressure corresponding to a diastolic pressure and a second pressure corresponding to a systolic pressure. In one aspect, this may include a diastolic pressure for a population of users and a second pressure corresponding to a systolic pressure for the population of users. While it is generally only necessary to span the systolic and diastolic pressure for the particular user who is calibrating the device, using a range of pressures that spans a broader representative population can improve the chances that a suitable range of pressures will be applied when calibrating the device for a current user, and lies within ranges that are easily achievable with tactile pressure. In another aspect, the range of forces may include a range of forces creating pressure on the user from less than 40 mmHg to greater than 150 mmHg, or a range of forces creating pressure on the user from less than 70 mmHg to greater than 140 mmHg. More generally, a lower bound for the range of pressures may be from 30 mmHg to 75 mmHg, and the higher bound for the range of pressures may be from 100 mmHg to 150 mmHg. In one aspect, these forces may be applied by manually applying a time-varying force to the wearable device while measuring the contact force between the wearable device and the user with the force sensor.

While systolic and diastolic blood pressures can be estimated using algorithms that interpret the shape and characteristics of the oscillometric envelope, the oscillometric envelope may also be used to extract parameters for a transfer function or other model that permits continuous monitoring of blood pressure, subject to other constraints. Thus in one aspect, the calibration data may also include a transfer function for an oscillometric envelope derived from the optical data acquired while applying a range of forces to the user with the wearable device. For example, U.S. Pat. App. No. 2013/006012 to Baron describes a use of the oscillometric method to calculate blood pressure, and Fortin, et al., “A novel art of continuous noninvasive blood pressure measurement,” Nature Communications (2021) describes a transfer function for an oscillometric envelope that can be used for continuous blood pressure monitoring. Each of the foregoing documents is incorporated herein by reference in its entirety. These oscillometric envelope techniques may be adapted for use with the systems and methods described herein to obtain calibrated blood pressure measurements from a wearable physiological monitor. For example, as distinguished from Fortin, which uses an actuator to maintain constant pressure while optically measuring blood flow, the current system contemplates the use of an elastic strap such as a wrist band of a wearable device to maintain a substantially constant pressure after calibration to facilitate continuous blood pressure monitoring based on optical data.

Other techniques measure changes in pulse shape in response to changes in applied pressure, and use this relationship to infer arterial pressure based on pulse shape under constant (or constrained) external pressure. For example, this may include calculating a K value for pulse shape under a range of pressures, as described for example in Yang, et al., “Pulse Wave K Value Averaging Computation and Pathological Diagnosis,” Journal of Computers, Vol. 8, No. 6, (June 2013), incorporated by reference herein in its entirety. These techniques may also or instead be used to analyze calibration data from the wearable device for blood pressure measurements and to obtain a model 1430 for continuous measurement of calibrated blood pressure from the wearable device. Thus in one aspect, the calibration data may include K values calculated for the optical signal while measuring the contact force between the wearable device and the user, e.g., over a range of pressures such as any of the pressure ranges described herein. Other measures of pulse shape based on, e.g., pulse symmetry and/or other pulse features, are also known in the art, and may be used to characterize changes in pulse shape in response to applied pressure. These techniques may be used in turn to calibrate a device for continuous calculation of blood pressure from optical data captured, e.g., with an optical signal passing through a radial artery.

According to the foregoing, obtaining calibration data as described herein may more generally include guiding the user through an application of varying pressures to the radial artery while capturing the optical data from a sensor positioned over the radial artery, or as a difference signal between a first optical signal passing through a radial artery and a second optical signal that is not passing through the radial artery, while also capturing force or pressure data from the force sensor. Where a second optical sensor is used, the second optical sensor may be used to remove non-radial artery artifacts from the optical signal of interest. In other embodiments, e.g., where the optical sensor over the radial artery provides a clean, useable signal independently, a second optical sensor may still be used, e.g., to periodically check that the optical sensor remains properly positioned over the radial artery.

In general, the calibration data may be used to create a model 1430 for calculating blood pressure based on optical data acquired from the calibration 1404. The model 1430 may then be used, e.g., with other data such as a measurement of strap tension, to continuously calculate a calibrated blood pressure for a wearer of the calibrated device. In general, the oscillometric envelope, K values, and other waveform analysis techniques can be used to compare an applied pressure to the arterial pressure. By measuring the applied force over a range of values that span the diastolic and systolic arterial pressures, a physical anchor can be provided to convert the results of the waveform analysis to a calibrated blood pressure (in mmHG). It will be understood that this conversion may be deployed, e.g., as a transfer function, a lookup table, or any other technique for mapping waveform characteristics to blood pressure in a manner that supports a model 1430 for continuous blood pressure calculation. It will also be understood that the actual tension of the strap (and/or contact force applied by the strap to a user's skin) may be used to measure the current force that is producing the waveform, and/or to convert a blood pressure measurement into a calibrated blood pressure measurement that corresponds to, e.g., cuff pressure or the like.

As shown in step 1412, the method 1400 may include measuring blood pressure, e.g., by acquiring calibrated blood pressure measurements. Once the wearable device has been positioned, and calibration data has been acquired to support the model 1430 for blood pressure calculation, continuous or intermittent blood pressure measurements may be acquired. This may, for example, include steps 1414-1418 below, which may be performed e.g., on a wearable device, or on a computing device associated with the wearable device, such as a server, a smart phone, a tablet, a laptop, or the like, or any combination of these.

As shown in step 1414, measuring blood pressure 1412 may include measuring a tension of the elastic strap in a suitable position, such as the position over the radial artery where blood pressure measurements are to be taken. This may include measuring the tension with any of the techniques described herein, such as by haptically stimulating the wearable device and measuring a dynamic response to the haptic stimulus to resolve a strap tension. In another aspect, a force sensor in the wearable device may be used to calculate pressure on the radial artery, e.g., by measuring the instantaneous contact force with the force sensor, and multiplying this by the surface area of the contact surface (e.g., the rigid contact surface of the housing, excluding the elastic strap that retains the housing in position) between the wearable device and the user's skin. In another aspect, the removable and replaceable force sensor that was used to calibrate the device may be left in position over an interval during which calibrated blood pressure measurements are desired, or a measurement of contact force may be taken with the force sensor as a single point of reference before removal.

Measuring tension may also or instead include measuring tension using any of the techniques described herein for evaluating optical and/or mechanical coupling, e.g., in order to detect substantial changes in the position or tension of the wearable device that might indicate that a recalibration is necessary or helpful for acquiring accurate blood pressure data.

As shown in step 1416, measuring blood pressure 1412 may include acquiring optical data such as pulse data from the optical sensor of the wearable device over an interval of time, e.g., an interval spanning one or more actual heartbeats or one or more typical heartbeats. For example, assuming a typical heartbeat rate of sixty beats per minute, and an interval of at least three heartbeats, this would suggest a measurement interval of at least three seconds, although longer or shorter intervals may also be used.

As shown in step 1418, measuring blood pressure 1412 may include calculating an instantaneous blood pressure for the user based on the pulse data, the tension of the elastic strap, and the calibration data, e.g., using any of the models and/or techniques for non-invasive blood pressure monitoring described herein. This may, for example, include summary statistics or metrics such as a mean blood pressure, a diastolic blood pressure, and a systolic blood pressure for one or more heartbeats, or this may include a time varying arterial pulse measurement, e.g., as a time series or other sequence of measurements over one or more heartbeats.

As shown in step 1420, measuring blood pressure 1412 may include any additional processing that might usefully be performed based on measurements of calibrated blood pressure. For example, this may include storing the blood pressure measurement(s) on the wearable device, and/or on a user computing device, or on a server coupled in a communicating relationship with the wearable device. This facilitates inspection, display, historical analysis, and the like, as well as comparison to other fitness metrics and data to identify relationships between blood pressure and other factors.

In another aspect, additional processing may include displaying blood pressure measurements. This may include a display of blood pressure over a short interval, e.g., as blood pressure changes over the course of individual heart beats and accompanying pressure waves. In another aspect, this may include a display of average blood pressure over time, e.g., by displaying diastolic and systolic blood pressure measurements on a per-minute, per-hour, or per-day basis, or any other suitable time scale based on user preferences, rate of data acquisition, and so forth. In another aspect, blood pressure may be used alone or in combination with other data to suggest lifestyle changes, generate coaching recommendations, and so forth.

According to the foregoing, there is also disclosed herein a system for non-invasive blood pressure measurement. In one aspect, the system includes a wearable device including an optical sensor, a force sensor, and a strap for securing the wearable device to a user; and one or more processors. The one or more processors may, for example, include one or more processors on the wearable device, one or more processors on a computing device associated with a user of the wearable device, and/or one or more processors on a server. The one or more processors may be configured, e.g., by non-transitory computer executable code stored in a memory, to: guide the user of the wearable device through a positioning process, based on a first set of optical data from the optical sensor, to locate the optical sensor over a radial artery of the user, guide the user of the wearable device through a calibration process, based on a second set of optical data from the optical sensor and concurrent force data from the force sensor, to obtain calibration data for calculating a calibrated blood pressure measurement with the wearable device, measure a tension of the strap, based on a third set of optical data from the optical sensor, the tension of the strap, and the calibration data, calculating a current blood pressure for the user over an interval, and calculate a diastolic blood pressure and a systolic blood pressure for the user over the interval.

In one aspect, the system may include a display configured to present the diastolic blood pressure and the systolic blood pressure to the user. In another aspect, the force sensor may include a removable and replaceable force sensor.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

1. A method comprising:
acquiring a first optical signal from a wearable device while the wearable device is placed for use on a body of a user;
applying a mechanical pressure to the wearable device with a mechanical actuator;
acquiring a second optical signal from the wearable device while applying the mechanical pressure;
acquiring a motion signal from the wearable device, at least a portion of the motion signal acquired while applying the mechanical pressure;
based on the first optical signal, the second optical signal, and the motion signal, calculating one or more physical parameters of a dynamic model for a mechanical system including the wearable device, a strap securing the wearable device to the user, and the mechanical actuator;
calculating a blood pressure of the user by applying the motion signal to the dynamic model; and
displaying the blood pressure to a user.

2. The method of claim 1, wherein the wearable device is at least one of: a physiological monitor, a photoplethysmography monitor, a heart rate monitor, and a wrist-worn monitor with a strap.

3-5. (canceled)

6. The method of claim 1, wherein the one or more physical parameters include a tension of a strap securing the wearable device to the user.

7. The method of claim 1, wherein the one or more physical parameters includes an elasticity of a tissue of the user in a measurement volume of the wearable device.

8. The method of claim 1, wherein the one or more physical parameters includes a spring constant for a system including the wearable device and a strap securing the wearable device to the user.

9. The method of claim 1, wherein the one or more physical parameters includes an elasticity of a tissue of the user adjacent to the wearable device.

10. The method of claim 1, wherein the mechanical actuator includes a haptic output device associated with the wearable device.

11. The method of claim 1, wherein applying mechanical pressure includes vibrating the wearable device with a haptic stimulus.

12. The method of claim 11, wherein the haptic stimulus includes a time varying haptic vibration.

13. The method of claim 11, wherein the haptic stimulus includes a haptic CHIRP output.

14. The method of claim 1, wherein the motion signal includes data from an accelerometer.

15. The method of claim 1, wherein the motion signal includes data from at least one of a three-axis accelerometer and a three-axis gyroscope.

16. The method of claim 1, wherein calculating one or more physical parameters includes evaluating at least one of the physical parameters with a machine learning model based on inputs from one or more sensors of the wearable device.

17. The method of claim 1, wherein at least one of the first optical signal and the second optical signal is acquired when the user is at rest.

18. The method of claim 1, wherein at least one of the first optical signal and the second optical signal includes a photoplethysmography signal.

19. The method of claim 1, further comprising testing tension on a strap of the wearable device by performing the steps of:
acquiring a baseline optical signal;
applying a haptic stimulus after acquiring the baseline optical signal;
acquiring a test optical signal during the haptic stimulus; and
analyzing the test optical signal for motion artifacts indicative of a suitable tension range for measuring blood pressure with the wearable device.

20. The method of claim 1, further comprising:
calculating a change in blood pressure based on a change in a pulse shape acquired by the wearable device from a first pulse acquired temporally proximal to measuring the motion signal from the wearable device to a second pulse acquired temporally distal to measuring the motion signal; and
calculating a blood pressure for the user based on the calculated blood pressure and the change in blood pressure.

21-40. (canceled)

41. A computer program product comprising computer executable code embodied in a non-transitory computer readable medium that, when executing on one or more devices, causes the one or more devices to perform the steps of:
acquiring a first optical signal from a wearable device while the wearable device is placed for use on a body of a user;
applying a mechanical pressure to the wearable device with a mechanical actuator;
acquiring a second optical signal from the wearable device while applying the mechanical pressure;
acquiring a motion signal from the wearable device, at least a portion of the motion signal acquired while applying the mechanical pressure;
based on the first optical signal, the second optical signal, and the motion signal, calculating one or more physical parameters of a dynamic model for a mechanical system including the wearable device, a strap securing the wearable device to the user, and the mechanical actuator;
calculating a blood pressure of the user by applying the motion signal to the dynamic model; and
displaying the blood pressure to a user.

42. A system comprising:
a wearable device including one or more motion sensors;
a strap securing the wearable device to a user;
a mechanical actuator; and
one or more processors implemented by computer executable code to perform the steps of:
acquiring a first optical signal from the wearable device while the wearable device is placed for use on a body of the user;
applying a mechanical pressure to the wearable device with the mechanical actuator;
acquiring a second optical signal from the wearable device while applying the mechanical pressure;
acquiring a motion signal from the one or more motion sensors of the wearable device, at least a portion of the motion signal acquired while applying the mechanical pressure;
based on the first optical signal, the second optical signal, and the motion signal, calculating one or more physical parameters of a dynamic model for a mechanical system including the wearable device, the strap, and the mechanical actuator;
calculating a blood pressure of the user by applying the motion signal to the dynamic model; and
displaying the blood pressure to a user.

43. The system of claim 42, wherein the one or more processors are disposed on at least one of the wearable device, a user device, and a remote server.
